(19) United States
(12) Patent Application Publication
(10) Pub. No.: US 2026/0102384 A1
(43) Pub. Date: Apr. 16, 2026

(54) USE OF PIPENDOXIFENE FOR TREATING INFECTIONS OF DNA VIRUSES

(71) Applicant: Model Medicines, Inc., San Diego, CA (US)

(72) Inventors: Daniel Haders, San Diego, CA (US); Tyler Umansky, San Diego, CA (US); Virgil Woods, San Diego, CA (US); David Garvey, San Diego, CA (US); George Nicola, San Diego, CA (US)

(21) Appl. No.: 19/419,109

(22) Filed: Dec. 15, 2025

Related U.S. Application Data
(63) Continuation of application No. PCT/US2025/036362, filed on Jul. 3, 2025.
(60) Provisional application No. 63/667,563, filed on Jul. 3, 2024.
(60) Provisional application No. 63/682,664, filed on Aug. 13, 2024.
(60) Provisional application No. 63/743,555, filed on Jan. 9, 2025.
(60) Provisional application No. 63/745,727, filed on Jan. 15, 2025.
(60) Provisional application No. 63/813,739, filed on May 29, 2025.
(60) Provisional application No. 63/813,736, filed on May 29, 2025.

(51) Int. Cl. A61K 31/454; A61P 31/20

(52) U.S. Cl. CPC A61K 31/454; A61P 31/20

(57) ABSTRACT

Disclosed are methods and compositions for treating infections caused by DNA viruses, including symptoms of the infections. The infections can be co-infections of the DNA viruses and other viruses. The methods, for example, can include administering pharmaceutical compositions comprising pipendoxifene disclosed herein to a patient in need.

### RELATED APPLICATIONS

The present application claims priority to U.S. Provisional Application No. 63/667,563, filed Jul. 3, 2024; U.S. Provisional Application No. 63/682,664, filed Aug. 13, 2024; U.S. Provisional Application No. 63/743,555, filed Jan. 9, 2025; U.S. Provisional Application No. 63/745,727, filed Jan. 15, 2025; U.S. Provisional Application No. 63/813,736, filed May 29, 2025; and U.S. Provisional Application No. 63/813,739, filed May 29, 2025. The entire contents of these applications are hereby expressly incorporated by reference in their entireties.

### BACKGROUND

### Field

The present disclosure generally relates to the treatment of DNA viral infections. More specifically, the disclosure describes methods for the treatment of a DNA viral infection and/or treatment or prevention of symptoms of a DNA viral infection by administering pharmaceutical compositions.

### Description of the Related Art

From the Justinian plague in the pre-modern times (500s A.D.), to the COVID-19 outbreak in 2019, the history of humankind can be charted along the timeline of pandemic outbreaks. When a viral pathogen is known, vaccines have been important in keeping outbreaks at bay, like contemporary influenza, in many, but not all, cases and more recently, managing the COVID-19 pandemic. Vaccine development is, however, a lengthy and sometimes an impossible task, usually initiated only after proof of a virus's pandemic potential. Moreover, vaccine storage and distribution can be an obstacle. Small-molecule antiviral drugs, on the other hand, can offer a quicker therapeutic approach, but often have limited activity spectrum. There is an urgent need for compositions and methods for preventing, delaying the onset of, or treating an inflammatory effect of an infection or a disease caused by a virus, e.g., DNA viruses.

### SUMMARY

Disclosed herein include methods for preventing, delaying the onset of, or treating an infection or a disease caused by a DNA virus. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease.

Disclosed herein include methods for preventing, delaying the onset of, or treating an inflammatory effect of an infection or a disease caused by a DNA virus. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect.

In some embodiments, the inflammatory effect comprises acute hepatitis, chronic hepatitis, liver swelling, liver damage, joint inflammation, muscle pain and weakness, or blood vessel problem. In some embodiments, the inflammatory effect comprises peritonsillar abscesses, acute bacterial sinusitis, suppurative lymph nodes, mastoiditis, and/or sialadenitis. In some embodiments, the inflammatory effect comprises respiratory failure, a sequela of respiratory failure, acute lung injury, or acute respiratory distress syndrome. In some embodiments, the sequela of respiratory failure comprises multi-organ failure. In some embodiments, the composition comprises a therapeutically or prophylactically effective amount of pipendoxifene or a pharmaceutically acceptable salt, ester, solvate, stereoisomer, tautomer, or prodrug thereof.

In some embodiments, the DNA virus is a double-stranded DNA virus. In some embodiments, the DNA virus is a Baltimore class VII (BCVII) virus, e.g., a hepadnavirus and/or a caulimovirus. In some embodiments, the DNA virus is a pararetrovirus. In some embodiments, the DNA virus is hepatitis B virus (HBV). In some embodiments, the DNA virus is a Baltimore class I (BCI) virus, e.g., Duplodnaviria, Monodnaviria, Singelaviria, or Varidnaviria. In some embodiments, the DNA virus is Epstein-Barr virus (EBV).

In some embodiments, the infection or the disease caused by the DNA virus is a liver disease, pneumonia, interstitial lung disease, pancreatitis, myocarditis, mononucleosis, cancer, systemic lupus erythematosus (SLE), Sjögren's syndrome, rheumatoid arthritis, and/or multiple sclerosis (MS). In some embodiments, the liver disease comprises cirrhosis and/or liver failure. In some embodiments, the cancer is liver cancer. In some embodiments, the cancer comprises Burkitt lymphoma (BL), gastric carcinoma, Hodgkin lymphoma (HL), NK/T cell lymphoma (NKTL), nasopharyngeal carcinoma (NPC), diffused large B cell lymphoma (DLBCL), HIV-associated lymphomas, and/or post-transplant lymphoproliferative disease (PTLD).

In some embodiments, the subject in need thereof is a subject that is suffering from the infection or the disease, or a subject that is at a risk for the infection or the disease. In some embodiments, the infection or the disease is in the respiratory tract of the subject. In some embodiments, the infection or the disease is in the liver of the subject. In some embodiments, the infection or the disease is in the B cells of the subject. In some embodiments, the subject is a subject that has been exposed to the DNA virus, is suspected to have been exposed to the DNA virus, or is at a risk of being exposed to the DNA virus. In some embodiments, the subject is co-infected with human immunodeficiency virus (HIV) and the DNA virus. In some embodiments, the subject is co-infected with hepatitis C virus (HCV) and the DNA virus. In some embodiments, the subject is co-infected with hepatitis D virus (HDV) and the DNA virus. In some embodiments, the subject is a mammal. In some embodiments, the subject is a human.

The method can comprise administering to the subject one or more additional antiviral agents. In some embodiments, at least one of the one or more additional antiviral agents is co-administered to the subject with the composition. In some embodiments, at least one of the one or more additional antiviral agents is administered to the subject before the administration of the composition, after the administration of the composition, or both. In some embodiments, the composition comprises one or more additional therapeutic agents. In some embodiments, the one or more additional therapeutic agents comprise one or more additional antiviral agents. In some embodiments, the one or more additional antiviral agents is selected from the group consisting of a nucleoside, a non-nucleoside analogue reverse-transcriptase inhibitor, a nucleotide analogue reverse-transcriptase inhibitor, and/or a sodium/bile acid cotransporter inhibitor.

In some embodiments, the composition is administered to the subject by intravenous administration, nasal administration, pulmonary administration, oral administration, parenteral administration, or nebulization. In some embodiments, the composition is in the form of powder, pill, tablet, microtablet, pellet, micropellet, capsule, capsule containing microtablets, liquid, aerosols, or nanoparticles. In some embodiments, the composition is in a formulation for oral or intravenous administration. In some embodiments, the composition is administered to the subject once, twice, or three times a day. In some embodiments, the composition is administered to the subject once every day, every two days, or every three days. In some embodiments, the composition is administered to the subject over the course of at least two weeks, at least three weeks, at least four weeks, or at least five weeks. In some embodiments, the composition is administered at a dose from about 125 mg/kg to about 375 mg/kg, optionally at a dose of 125 mg/kg, 250 mg/kg, or 375 mg/kg.

The method can comprise measuring the viral titer of the DNA virus in the subject before administering the composition to the subject, after administering the composition to the subject, or both. In some embodiments, the viral titer is lung bulk virus titer or blood viral titer. In some embodiments, administrating the composition results in reduction of the viral titer of the DNA virus in the subject as compared to that in the subject before administration of the composition. The method can comprise determining global virus distribution in an organ or a tissue of the subject. The method can comprise determining global virus distribution in lungs of the subject and/or the blood of the subject. The method can comprise measuring a neutrophil density within the lungs of the subject. In some embodiments, administering the composition results in reduction of the neutrophil density within the lungs of the subject as compared to that in the subject before administration of the composition. The method can comprise measuring a total necrotized cell count within the lungs of the subject. In some embodiments, administering the composition results in reduction of the total necrotized cell count in the subject as compared to that in the subject before administration of the composition. The method can comprise measuring a total protein level within the lungs of the subject. In some embodiments, administering the composition results in reduction of the total protein level within the lungs of the subject as compared to that in the subject before administration of the composition. The method can comprise measuring liver enzyme levels in the blood of the subject. In some embodiments, the liver enzymes comprise alanine aminotransferase (ALT) and aspartate aminotransferase (AST). In some embodiments, administering the composition results in reduction of the liver enzyme levels in the blood of the subject as compared to that in the subject before administration of the composition. The method can comprise measuring albumin level in the blood of the subject. In some embodiments, administering the composition results in increase of the albumin level in the blood of the subject as compared to that in the subject before administration of the composition.

The method can comprise measuring HBV DNA and/or anti-HBV antibody in the blood of the subject. In some embodiments, administering the composition results in reduction of the HBV DNA and/or anti-HBV antibody in the subject as compared to that in the subject before administration of the composition. The method can comprise measuring EBV DNA and/or anti-EBV antibody in the blood of the subject. In some embodiments, administering the composition results in reduction of the EBV DNA and/or anti-EBV antibody in the subject as compared to that in the subject before administration of the composition.

In some embodiments, the growth inhibition IC50 of the compound to estrogen receptor of the subject is greater than 0.1 μM. In some embodiments, the growth inhibition IC50 of the compound to estrogen receptor of the subject is greater than 1 μM. In some embodiments, the growth inhibition IC50 of the compound to estrogen receptor of the subject is greater than 10 μM.

Disclosed herein includes a method for preventing, delaying the onset of, or treating a HBV and HDV co-infection or a disease caused by a HBV and HDV co-infection, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the HBV and HDV co-infection or the disease.

Also disclosed herein includes a method for preventing, delaying the onset of, or treating an inflammatory effect of a HBV and HDV co-infection or a disease caused by a HBV and HDV co-infection, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect. The inflammatory effect can comprises, e.g., acute hepatitis, chronic hepatitis, liver swelling, liver damage, joint inflammation, muscle pain and weakness, or blood vessel problem.

In some embodiments, the disease caused by the HBV and HDV co-infection is a liver disease, optionally the liver disease comprises cirrhosis and/or liver failure. In some embodiments, the disease caused by the HBV and HDV co-infection is liver cancer.

In some embodiments, the subject in need thereof is a subject that is suffering from the HBV and HDV co-infection or the disease, or a subject that is at a risk for the HBV and HDV co-infection or the disease. In some embodiments, the HBV and HDV co-infection or the disease is in the liver of the subject. The subject can be, e.g., a subject that has been exposed to HBV and/or HDV, is suspected to have been exposed to HBV and/or HDV, or is at a risk of being exposed to HBV and/or HDV. In some embodiments, the subject has or is at risk of having acute HDV and/or chronic HDV. The subject can be, e.g., a mammal, optionally the subject is a human.

In some embodiments, the method comprises administering to the subject one or more additional antiviral agents. In some embodiments, at least one of the one or more additional antiviral agents is co-administered to the subject with the composition. In some embodiments, at least one of the one or more additional antiviral agents is administered to the subject before the administration of the composition, after the administration of the composition, or both. The composition can comprise one or more additional therapeutic agents (e.g., one or more additional antiviral agents). The one or more additional antiviral agents can be, e.g., a nucleoside, a non-nucleoside analogue reverse-transcriptase inhibitor, a nucleotide analogue reverse-transcriptase inhibitor, an interferon and/or a sodium/bile acid cotransporter inhibitor.

The composition can be, e.g., administered to the subject by intravenous administration, nasal administration, pulmonary administration, oral administration, parenteral administration, or nebulization. The composition can be, e.g., in the form of powder, pill, tablet, microtablet, pellet, micropellet, capsule, capsule containing microtablets, liquid, aerosols, or nanoparticles.

In some embodiments, the composition is in a formulation for oral or intravenous administration. In some embodiments, the composition is administered to the subject once, twice, or three times a day. In some embodiments, the composition is administered to the subject once every day, every two days, or every three days. In some embodiments, the composition is administered to the subject over the course of at least two weeks, at least three weeks, at least four weeks, or at least five weeks. In some embodiments, the composition is administered at a dose from about 125 mg/kg to about 375 mg/kg of pipendoxifene, optionally at a dose of 125 mg/kg, 250 mg/kg, or 375 mg/kg of pipendoxifene.

In some embodiments, the method further comprises measuring the viral titer of HBV and/or HDV in the subject before administering the composition to the subject, after administering the composition to the subject, or both, optionally the viral titer is blood viral titer. In some embodiments, the method comprises administrating the composition results in reduction of the viral titer of the HBV and/or HDV in the subject as compared to that in the subject before administration of the composition. In some embodiments, the method further comprises determining global HBV and/or HDV distribution in an organ or a tissue of the subject; and optionally the method comprises determining global virus distribution in the blood of the subject. In some embodiments, the method further comprises measuring liver enzyme levels in the blood of the subject, optionally the liver enzymes comprise alanine aminotransferase (ALT) and aspartate aminotransferase (AST), and further optionally administering the composition results in reduction of the liver enzyme levels in the blood of the subject as compared to that in the subject before administration of the composition. In some embodiments, the method further comprises measuring albumin level in the blood of the subject, optionally administering the composition results in increase of the albumin level in the blood of the subject as compared to that in the subject before administration of the composition. In some embodiments, the method further comprises measuring HBV and/or HDV DNA in the blood of the subject, optionally administering the composition results in reduction of the HBV and/or HDV DNA in the subject as compared to that in the subject before administration of the composition. In some embodiments, the method further comprises measuring HBV and/or HDV antibody in the blood of the subject, optionally administering the composition results in reduction of the HBV and/or HDV antibody in the subject as compared to that in the subject before administration of the composition

Disclosed herein include compositions for use in preventing, delaying the onset of, or treating an infection or a disease caused by a DNA virus. In some embodiments, the composition comprises pipendoxifene or a pharmaceutically acceptable salt, ester, solvate, stereoisomer, tautomer, or prodrug thereof, thereby preventing, delaying the onset of, or treating the infection or the disease.

Disclosed herein include compositions for use in preventing, delaying the onset of, or treating an inflammatory effect of an infection or a disease caused by a DNA virus. In some embodiments, the composition comprises pipendoxifene or a pharmaceutically acceptable salt, ester, solvate, stereoisomer, tautomer, or prodrug thereof, thereby preventing, delaying the onset of, or treating the infection or the disease.

In some embodiments, the inflammatory effect comprises acute hepatitis, chronic hepatitis, liver swelling, liver damage, joint inflammation, muscle pain and weakness, and/or blood vessel problem. In some embodiments, the inflammatory effect comprises peritonsillar abscesses, acute bacterial sinusitis, suppurative lymph nodes, mastoiditis, and/or sialadenitis. In some embodiments, the inflammatory effect comprises respiratory failure, a sequela of respiratory failure, acute lung injury, and/or acute respiratory distress syndrome. In some embodiments, the sequela of respiratory failure comprises multi-organ failure. In some embodiments, the composition comprises a therapeutically or prophylactically effective amount of pipendoxifene, or a pharmaceutically acceptable salt, ester, solvate, stereoisomer, tautomer, or prodrug thereof.

In some embodiments, the DNA virus is a double-stranded DNA virus. In some embodiments, the DNA virus is a Baltimore class VII (BCVII) virus, e.g., a hepadnavirus and/or a caulimovirus. In some embodiments, the DNA virus is a pararetrovirus. In some embodiments, the DNA virus is hepatitis B virus (HBV). In some embodiments, the DNA virus is a Baltimore class I (BCI) virus, e.g., Duplodnaviria, Monodnaviria, Singelaviria, or Varidnaviria. In some embodiments, the DNA virus is Epstein-Barr virus (EBV). In some embodiments, the infection or the disease caused by the DNA virus is a liver disease, pneumonia, interstitial lung disease, pancreatitis, myocarditis, mononucleosis, cancer, systemic lupus erythematosus (SLE), Sjögren's syndrome, rheumatoid arthritis, and/or multiple sclerosis (MS). In some embodiments, the liver disease comprises cirrhosis and/or liver failure. In some embodiments, the cancer is liver cancer. In some embodiments, the cancer comprises Burkitt lymphoma (BL), gastric carcinoma, Hodgkin lymphoma (HL), NK/T cell lymphoma (NKTL), nasopharyngeal carcinoma (NPC), diffused large B cell lymphoma (DLBCL), HIV-associated lymphomas, and/or post-transplant lymphoproliferative disease (PTLD).

In some embodiments, the composition is a pharmaceutical composition comprising pipendoxifene or a pharmaceutically acceptable salt, ester, solvate, stereoisomer, tautomer, or prodrug thereof, and one or more pharmaceutically acceptable excipients. In some embodiments, the composition comprises one or more additional therapeutic agents. In some embodiments, the one or more additional therapeutic agents comprise one or more antiviral agents. In some embodiments, the one or more antiviral agents is selected from the group consisting of a nucleoside, a non-nucleoside analogue reverse-transcriptase inhibitor, a nucleotide analogue reverse-transcriptase inhibitor, and/or a sodium/bile acid cotransporter inhibitor. In some embodiments, the composition is in the form of powder, pill, tablet, microtablet, pellet, micropellet, capsule, capsule containing microtablets, liquid, aerosols, or nanoparticles. In some embodiments, the composition is in a formulation for oral or intravenous administration.

Disclosed herein includes a composition for use in preventing, delaying the onset of, or treating a HBV and HDV co-infection or a disease caused by a HBV and HDV co-infection, wherein the composition comprises pipendoxifene or a pharmaceutically acceptable salt, ester, solvate, stereoisomer, tautomer, or prodrug thereof, thereby preventing, delaying the onset of, or treating the HBV and HDV co-infection or the disease. Also disclosed includes a composition for use in preventing, delaying the onset of, or treating an inflammatory effect of a HBV and HDV co-infection or a disease caused by a HBV and HDV co-infection or a disease caused by a HBV and HDV co-infection, wherein the composition comprises pipendoxifene or a pharmaceutically acceptable salt, ester, solvate, stereoisomer, tautomer, or prodrug thereof, thereby preventing, delaying the onset of, or treating the HBV and HDV co-infection or the disease. The inflammatory effect can, e.g., comprise acute hepatitis, chronic hepatitis, liver swelling, liver damage, joint inflammation, muscle pain and weakness, and/or blood vessel problem.

In some embodiments, the composition comprises a therapeutically or prophylactically effective amount of pipendoxifene, or a pharmaceutically acceptable salt, ester, solvate, stereoisomer, tautomer, or prodrug thereof. In some embodiments, the disease caused by the HBV and HDV co-infection is a liver disease, optionally the liver disease comprises cirrhosis and/or liver failure. In some embodiments, the disease caused by the HBV and HDV co-infection is liver cancer.

The composition, in some embodiments, is a pharmaceutical composition comprising pipendoxifene or a pharmaceutically acceptable salt, ester, solvate, stereoisomer, tautomer, or prodrug thereof, and one or more pharmaceutically acceptable excipients. The composition can comprise, e.g., one or more additional therapeutic agents (e.g., one or more antiviral agents). In some embodiments, the one or more antiviral agents is a nucleoside, a non-nucleoside analogue reverse-transcriptase inhibitor, a nucleotide analogue reverse-transcriptase inhibitor, an interferon, and/or a sodium/bile acid cotransporter inhibitor. In some embodiments, the composition is in the form of powder, pill, tablet, microtablet, pellet, micropellet, capsule, capsule containing microtablets, liquid, aerosols, or nanoparticles. In some embodiments, the composition is in a formulation for oral or intravenous administration.

Disclosed herein include kits. In some embodiments, the kit comprises pipendoxifene or a pharmaceutically acceptable salt, ester, solvate, stereoisomer, tautomer, or prodrug thereof; and a label indicating that the kit is for preventing, delaying the onset of, or treating an infection or a disease caused by a DNA virus; or a label indicating that the kit is for preventing, delaying the onset of, or treating an inflammatory effect of an infection or a disease caused by a DNA virus. In some embodiments, the DNA virus is a Baltimore class VII (BCVII) virus, e.g., a hepadnavirus and/or a caulimovirus. In some embodiments, the DNA virus is a pararetrovirus. In some embodiments, the DNA virus is hepatitis B virus (HBV). In some embodiments, the DNA virus is a Baltimore class I (BCI) virus, e.g., Duplodnaviria, Monodnaviria, Singelaviria, or Varidnaviria. In some embodiments, the DNA virus is Epstein-Barr virus (EBV).

As disclosed herein, pipendoxifene has a formula of:

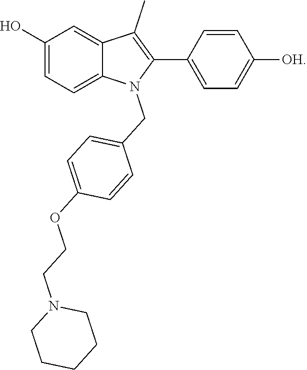 [SMILES: Cc1c(-c2ccc(O)cc2)n(Cc2ccc(OCCN3CCCCC3)cc2)c2ccc(O)cc12]

### BRIEF DESCRIPTION OF THE DRAWINGS

FIG. 1A-FIG. 1C display non-limiting exemplary data showing extracellular HBV DNA quantification by qPCR in HepAD38 cells following 10 days of MDL-001 treatment. Data series correspond to Experiment 1 (FIG. 1A; 10% FBS, treatments on Days 1, 4 and 8; 0-10 000 nM MDL-001), Experiment 2 (FIG. 1B; 10% FBS, daily treatments; 0-2 500 nM MDL-001), and Experiment 3 (FIG. 1C; 5% FBS, treatments on Days 1, 4 and 8; 0-10 000 nM MDL-001). Day 10 IC50 values are reported, including FBS corrected values for 10% FBS conditions and a correction estimate for the 5% condition (see Example 4 below).

FIG. 2A-FIG. 2C display non-limiting exemplary data showing secreted HBsAg levels measured by ELISA in HepAD38 cells after 10 days of MDL-001 exposure. Treatment regimens are as in FIG. 1A-FIG. 1C (FIG. 2A=Experiment 1; FIG. 2B=Experiment 2; FIG. 2C=Experiment 3). Day 10 IC50 values are reported, including FBS corrected values for 10% FBS conditions and a correction estimate for the 5% condition (see Example 4 below).

FIG. 3A-FIG. 3C display non-limiting exemplary dose-response curves of HCV/HBV co-infected Huh7.5.1 cells treated with MDL-001. Huh7.5.1 cells co-infected on Day 0 with HCV JFH-1 and HBV were treated in triplicate with MDL-001 (0-25 000 nM) on Days 0, 3, and 6. Supernatants harvested on Days 3 (FIG. 3A), 6 (FIG. 3B), and 9 (FIG. 3C) were analyzed by one-step RT-qPCR for HCV and by qPCR for HBV DNA. Viral levels are normalized to the DMSO control and expressed as mean±SD. Remdesivir and sofosbuvir served as antiviral controls.

FIG. 4A-FIG. 4B display non-limiting exemplary data related to HBV response to MDL-001 treatment in a mouse in vivo model of infection. Readout is HBV DNA copies/mL in plasma, plus an extra measurement of liver HBV DNA copies/mL. Values are expressed as normalized percentages to the vehicle control on each day of infection. Individual vehicle group values are shown surrounding their average value, which is set to 100% for each day's measurements across groups. Statistical differences at Day 10 were assessed by one-way ANOVA on log₁₀-transformed plasma and liver viral loads (Vehicle vs. treatment), yielding P<0.005 (**), P<0.001 (***), or P<0.0001 (****) for indicated groups. The plasma Day 13, plasma Day 16, and liver Day 16 values were normalized to extrapolated vehicle values from the growth curve detailed in FIG. 4B. The vehicle group's raw data in virus copies/mL is shown in FIG. 4B. Plasma values for Day 13 and Day 16 were calculated based on Day 16 measurements. Liver values for the vehicle group on Day 16 were extrapolated by applying the calculated Day 16/Day 10 plasma ratio to the Day 10 liver values of the Vehicle control group.

FIG. 5 displays non-limiting exemplary data related to HDV antigen response to MDL-001 treatment in an HBV+HDV co-infection model in hNTCP-Huh7 cell culture supernatant, with either daily dosing or doses every 3 days. Conditions were either 3% or 10% FBS. Results show responses at Day 10 of each condition.

FIG. 6 displays non-limiting exemplary data related to HDV Antigen response to MDL-001 treatment in an HBV+HDV co-infection model in hNTCP-Huh7 cell lysate, with either daily dosing or doses every 3 days. Conditions were either 3% or 10% FBS. Results show responses at Day 10 of each condition.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods and compositions for use for preventing, delaying the onset of, or treating an infection or a disease caused by a DNA virus. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease.

Disclosed herein include methods and compositions for use for preventing, delaying the onset of, or treating an inflammatory effect of an infection or a disease caused by a DNA virus. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect.

Disclosed herein include methods and compositions for use for preventing, delaying the onset of, or treating a hepatitis B virus (HBV) and hepatitis D virus (HDV) co-infection or a disease caused by a HBV and HDV co-infection, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the HBV and HDV co-infection or the disease.

Disclosed herein include methods and compositions for use for preventing, delaying the onset of, or treating an inflammatory effect of a hepatitis B virus (HBV) and hepatitis D virus (HDV) co-infection or a disease caused by a HBV and HDV co-infection, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect.

Disclosed herein include kits. In some embodiments, the kit comprises pipendoxifene or a pharmaceutically acceptable salt, ester, solvate, stereoisomer, tautomer, or prodrug thereof; and a label indicating that the kit is for preventing, delaying the onset of, or treating an infection or a disease caused by a DNA virus; or a label indicating that the kit is for preventing, delaying the onset of, or treating an inflammatory effect of an infection or a disease caused by a DNA virus.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” means plus or minus 5% of the provided value.

As used herein, “compound”, “compounds”, “chemical entity”, and “chemical entities” refer to a compound encompassed by the generic formula disclosed herein, any subgenus of those generic formula, and any forms of the compounds within the generic and subgeneric formula, including the pharmaceutically acceptable salts, esters, solvates, racemates, stereoisomers, tautomers, and prodrugs of the compound or compounds.

As used herein, “solvate” or “solvates” of a compound refer to those compounds, as defined above, which are bound to a stoichiometric or non-stoichiometric amount of a solvent. Solvates of a compound includes solvates of all forms of the compound. In some embodiments, solvents are volatile, non-toxic, and/or acceptable for administration to humans in trace amounts. Suitable solvates include water.

As used herein, “racemates” refers to a mixture of enantiomers. In some embodiments, the compounds, or pharmaceutically acceptable salts thereof, can be enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

As used herein, “stereoisomer” or “stereoisomers” refer to compounds that differ in the chirality of one or more stereocenters. Stereoisomers include enantiomers and diastereomers.

As used herein, “tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable” as used herein means approved by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound that is pharmaceutically acceptable and that possesses the desired pharmacological activity of the parent compound. Such salts include but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound disclosed herein is administered.

“Prodrug” refers to a derivative of a drug molecule that requires a transformation within the body to release the active drug. Prodrugs are frequently (though not necessarily) pharmacologically inactive until converted to the parent drug. Typically, prodrugs are designed to overcome pharmaceutical and/or pharmacokinetically based problems associated with the parent drug molecule that would otherwise limit the clinical usefulness of the drug.

“Promoiety” refers to a form of protecting group that when used to mask a functional group within a drug molecule converts the drug into a prodrug. Typically, the promoiety will be attached to the drug via bond(s) that are cleaved by enzymatic or non-enzymatic means in vivo. Ideally, the promoiety is rapidly cleared from the body upon cleavage from the prodrug.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e. Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place.

As used herein, “administration” or “administering” refers to a method of giving a dosage of a pharmaceutically active ingredient to a vertebrate.

As used herein, a “dosage” refers to the combined amount of the active ingredients (e.g., berzosertib and/or pipendoxifene).

As used herein, “therapeutically effective amount” or “pharmaceutically effective amount” is meant an amount of therapeutic agent, which has a therapeutic effect. The dosages of a pharmaceutically active ingredient that are useful in treatment are therapeutically effective amounts. Thus, as used herein, a therapeutically effective amount means an amount of therapeutic agent, which produces the desired therapeutic effect as judged by clinical trial results and/or model animal studies.

As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire). “Treat,” “treatment,” or “treating,” as used herein refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, EC₅₀ is the value of a graded dose response curve that represents the concentration of a compound where 50% of its maximal effect is observed.

As used herein, CC₅₀ is the 50% cytotoxic concentration defined as the compound's concentration (μg/mL) required for the reduction of cell viability by 50%.

As used herein, SI=CC₅₀/EC₅₀. The selectivity index (SI) is a ratio that measures the window between cytotoxicity and antiviral activity by dividing the CC₅₀ value into the EC₅₀ value. The higher the SI ratio, the theoretically more effective and safe a drug would be during in vivo treatment for a given viral infection.

“Individual” as used herein refers to a person, human adult or child, mammal, or non-human primate.

“IC50” as used herein refers to the molar concentration of a compound, for pipendoxifene, which binds 50% of receptor related to a viral infection in vitro.

“Ki” as used herein refers to the kinetic inhibition constant in molar concentration units that denotes the affinity of a compound, e.g., pipendoxifene, for the receptor related to a viral infection as measured by a binding assay or as calculated from the IC50 value using the Cheng-Prusoff equation.

“Patient” as used herein refers to a mammal, e.g., a human, mouse, rat, guinea pig, dog, cat, horse, cow, pig, or non-human primate, such as a monkey, chimpanzee, baboon or rhesus.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Treating” or “treatment” of any disease or disorder as used herein, refers, in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” as used herein, means the amount of a compound that, when administered to an individual for treating a disease, is sufficient to effect such treatment for the disease or to achieve the desired clinical response. The “therapeutically effective amount” will vary depending on the compound, the disease and its severity and the age, weight, etc., of the patient to be treated.

As used herein, a “dosage” refers to an amount of therapeutic agent administered to a patient.

As used herein, a “daily dosage” refers to the total amount of therapeutic agent administered to a patient in a day.

As used herein, the term “therapeutic agent” means a substance that is effective in the treatment of a disease or condition.

Pipendoxifene exhibits high binding ability towards RNA-dependent RNA polymerase (RdRp) and specifically its Thumb-1 site, an allosteric subdomain of RdRp. RdRp is a polymerase that catalyzes the replication of RNA from an RNA template and is a vital enzyme for RNA viruses' replication/transcription complex. This enzyme synthesizes a full-length negative-strand RNA template that can subsequently be used to replicate and transcribe the viral genome. RdRp is an essential protein encoded in the genomes of most RNA-containing viruses that lack a DNA stage. RdRp's crucial role in the life cycle of RNA viruses has led to its targeted inhibition for a number of viral infections such as hepatitis C virus, Zika virus, and coronaviruses. The core RdRp domain consists of the thumb, palm and the fingers subdomains that are primarily involved in template binding, polymerization, nucleoside triphosphate (NTP) entry and associated functions. The palm subdomain is at the junction of the fingers and the thumb subdomains and houses most of the structurally conserved elements involved in catalysis. The catalytic aspartates and the RNA Recognizing Motif (RRM) comprising three β-strands are present in the palm subdomain. The thumb subdomain harbors residues that are involved in packing against the template RNA and stabilizing the initiating NTPs on the template. This subdomain also facilitates the translocation of template following polymerization by accommodating large conformational rearrangements.

MDL-001 (Pipendoxifene) has a chemical structure of:

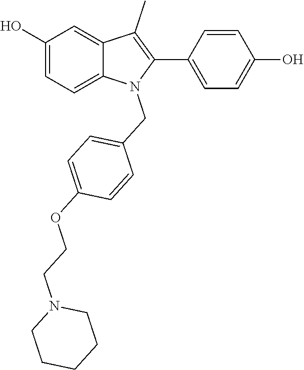 [SMILES: Cc1c(-c2ccc(O)cc2)n(Cc2ccc(OCCN3CCCCC3)cc2)c2ccc(O)cc12]

As provided herein, it was surprisingly found that pipendoxifene can inhibit DNA viral infections and viral infections caused by co-infections of DNA viruses and other viruses.

### Methods for Treating Viral Infection

Disclosed herein include methods and compositions for preventing, delaying the onset of, or treating an infection or a disease caused by a DNA virus. In some embodiments, the method comprises administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease. Disclosed herein include methods for preventing, delaying the onset of, or treating an inflammatory effect of an infection or a disease caused by a DNA virus. In some embodiments, the method comprises: administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect.

The inflammatory effect can comprise acute hepatitis, chronic hepatitis, liver swelling, liver damage, joint inflammation, muscle pain and weakness, or blood vessel problem. The inflammatory effect can comprise peritonsillar abscesses, acute bacterial sinusitis, suppurative lymph nodes, mastoiditis, and/or sialadenitis. In some embodiments, the inflammatory effect comprises respiratory failure, a sequela of respiratory failure, acute lung injury, or acute respiratory distress syndrome. In some embodiments, the sequela of respiratory failure comprises multi-organ failure.

A pro-inflammatory cytokine or a pro-inflammatory mediator can be an immuno-regulatory cytokine that favor inflammation. Pro-inflammatory cytokines that are generally responsible for early immune responses include IL-1, IL-6, and TNF-α. IL-1, IL-6, and TNF-α are also considered endogenous pyrogens as they contribute to increasing body temperature. Other examples of pro-inflammatory cytokines or pro-inflammatory mediators include IL-8, IL-11, IL-12, IL-18, GM-CSF, IFN-γ, TGF-β, leukemia inhibitory factors (LIF), oncostatin M (OSM), and a variety of chemokines that attract inflammatory cells. A pro-inflammatory cytokine generally up-regulates or increases the synthesis of secondary pro-inflammatory mediators and other pro-inflammatory cytokines by immune cells. In addition, pro-inflammatory cytokines can stimulate production of acute phase proteins that mediate inflammation and attract inflammatory cells. The method can comprise an at least, or at least about, 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) reduction in the level of one or more of interferon-γ (IFNγ), IL-1, IL-6, transforming growth factor-α (TGFα), transforming growth factor-β (TGFβ), CCL2, CXCL10, IL-11, IL-12, IL-18, GM-CSF, CXCL9 and IL-8 in the subject. The compositions and methods provided herein can reduce the production and/or amount of a pro-inflammatory cytokine and/or a pro-inflammatory mediator in the lung and/or serum by at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used.

The inflammatory effect can comprise respiratory failure, a sequela of respiratory failure, acute lung injury, or acute respiratory distress syndrome. The sequela of respiratory failure can comprise multi-organ failure. As used herein, the terms “inflammation” and “inflammatory response” shall be given their ordinary meaning, and also include immune-related responses and/or allergic reactions to a physical, chemical, or biological stimulus. Measuring inflammation (e.g. lung or liver inflammation) can comprise measuring the level of a pro-inflammatory cytokine, an anti-inflammatory cytokine, or a combination of pro-inflammatory cytokines and anti-inflammatory cytokines.

Inflammation (e.g. related to the liver) can comprise acute hepatitis, chronic hepatitis, liver swelling, liver damage, joint inflammation, muscle pain and weakness, or blood vessel problem. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of one or more of acute hepatitis, chronic hepatitis, liver swelling, liver damage, joint inflammation, muscle pain and weakness, or blood vessel problem.

Inflammation can comprise peritonsillar abscesses, acute bacterial sinusitis, suppurative lymph nodes, mastoiditis, and/or sialadenitis. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of one or more of peritonsillar abscesses, acute bacterial sinusitis, suppurative lymph nodes, mastoiditis, and/or sialadenitis.

Inflammation (e.g. lung inflammation) can comprise mast cell degranulation, plasma extravasation, and bronchoconstriction. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of one or more of mast cell degranulation, plasma extravasation, and bronchoconstriction. In some embodiments of the methods and compositions provided herein, lymphopenia and/or mononuclear cell infiltration in the lungs is reduced by at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein).

The composition can comprise a therapeutically or prophylactically effective amount of pipendoxifene. The subject in need can be a subject that can be suffering from the infection or the disease, or a subject that can be at a risk for the infection or the disease. The infection or the disease can be in the respiratory tract of the subject. The infection or the disease can be in the liver of the subject. The infection or the disease can be in the B cells of the subject. In some embodiments, the subject is a subject that has been exposed to the DNA virus, is suspected to have been exposed to the DNA virus, or is at a risk of being exposed to the DNA virus. The subject can be a mammal. The subject can be a human. The infection or the disease caused by the DNA virus can be a liver disease, pneumonia, interstitial lung disease, pancreatitis, myocarditis, mononucleosis, cancer, systemic lupus erythematosus (SLE), Sjögren's syndrome, rheumatoid arthritis, and/or multiple sclerosis (MS). The liver disease can comprise cirrhosis and/or liver failure. The cancer can be liver cancer. The cancer can comprise Burkitt lymphoma (BL), gastric carcinoma, Hodgkin lymphoma (HL), NK/T cell lymphoma (NKTL), nasopharyngeal carcinoma (NPC), diffused large B cell lymphoma (DLBCL), HIV-associated lymphomas, and/or post-transplant lymphoproliferative disease (PTLD).

In some embodiments, the virus is a single stranded or is a double stranded DNA species. In some embodiments, the virus is a member of any one of seven Baltimore classifications. Developed and proposed by David Baltimore, the Baltimore classifications divide viruses into major groups based on their genomic structure and replication strategy. In some aspects, group I viruses comprise a double-stranded DNA genome, such as viruses belonging to Herpesviridae (e.g., Herpes simplex virus type 1), Adenoviridae (e.g., human Adenovirus), and Papovaviridae. In some aspects, group II viruses comprise a single-stranded DNA genome, such as viruses belonging to Parvoviridae. In some embodiments, the binding agent or particle inhibits the replication of Baltimore Group III viruses. In some aspects, group III viruses comprise a double-stranded RNA genome, such as viruses belonging to Reoviridae and Birnaviridae. In some aspects, group IV viruses comprise a single-stranded RNA genome of the positive sense, such as viruses belonging to Coronaviridae, Flavivirdae (e.g., Dengue virus, West Nile virus, and Hepatitis C virus), Togaviridae (e.g., Chikungunya virus), and Picornaviridae. In some aspects, group V viruses comprise a single-stranded RNA genome of the negative sense, such as viruses belonging to Orthomyxoviridae (e.g., Influenza viruses), Paramyxoviridae, Filoviridae, and Rhabdoviridae. In some aspects, group VII viruses comprise a double-stranded RNA genome that requires and RNA intermediate, such as pararetroviruses (e.g., Hepatitis B). Thus, in some embodiments, the DNA virus is a class I, class II, or class VII virus. The DNA virus can be a double-stranded DNA virus. The DNA virus can be a Baltimore class VII (BCVII) virus, e.g., a hepadnavirus and/or a caulimovirus. The DNA virus can be a pararetrovirus. The DNA virus can be HBV. The DNA virus can be a Baltimore class I (BCI) virus, e.g., Duplodnaviria, Monodnaviria, Singelaviria, or Varidnaviria. The DNA virus can be Epstein-Barr virus (EBV).

Hepatitis B virus (HBV) is a partially double-stranded DNA virus, a species of the genus Orthohepadnavirus and a member of the Hepadnaviridae family of viruses. This virus causes the disease hepatitis B. Hepatitis B can be acute and later become chronic, leading to other diseases and health conditions. In addition to causing hepatitis, infection with HBV can lead to cirrhosis and hepatocellular carcinoma. The Epstein-Barr virus (EBV), also known as human herpesvirus 4 (HHV-4), is one of the nine known human herpesvirus types in the herpes family, and is one of the most common viruses in humans. EBV is a double-stranded DNA virus. EBV is the first identified oncogenic virus, a virus that can cause cancer. EBV establishes permanent infection in human B cells. It uncommonly causes infectious mononucleosis and is also tightly linked to many malignant diseases (cancers and autoimmune diseases).

The method can comprise administering to the subject one or more additional antiviral agents. At least one of the one or more additional antiviral agents can be co-administered to the subject with the composition. At least one of the one or more additional antiviral agents can be administered to the subject before the administration of the composition, after the administration of the composition, or both. The composition can comprise one or more additional therapeutic agents. The one or more additional therapeutic agents comprise one or more antiviral agents. In some embodiments, the one or more additional antiviral agents is selected from the group consisting of a nucleoside, a non-nucleoside analogue reverse-transcriptase inhibitor, a nucleotide analogue reverse-transcriptase inhibitor, and/or a sodium/bile acid cotransporter inhibitor. In some embodiments, the one or more additional antiviral agents is selected from a nucleoside, a non-nucleoside analogue reverse-transcriptase inhibitor, a nucleotide analogue reverse-transcriptase inhibitor, an interferon and a sodium/bile acid cotransporter inhibitor.

The composition can be a pharmaceutical composition comprising pipendoxifene and one or more pharmaceutically acceptable excipients. The composition can be administered to the subject by intravenous administration, nasal administration, pulmonary administration, oral administration, parenteral administration, or nebulization. The composition can be in the form of powder, pill, tablet, microtablet, pellet, micropellet, capsule (including capsule containing microtablets), liquid, aerosols, or nanoparticles. The composition can be in a formulation for intravenous administration. Compositions of the disclosure can also be used prophylactically for preventing, delaying the onset of, or treating an infection or a disease or inflammation caused by a DNA virus. The prophylactically effective amount of a compound (e.g., pipendoxifene) of the disclosure can be any therapeutically effective amount of a compound described herein.

The compositions of the disclosure can be administered via any suitable route. Potential routes of administration include without limitation oral, parenteral (including intramuscular, subcutaneous, intradermal, intravascular, intravenous, intraarterial, intramedullary and intrathecal), intracavitary, intraperitoneal, and topical (including dermal/epicutaneous, transdermal, mucosal, transmucosal, intranasal (e.g., by nasal spray or drop), intraocular (e.g., by eye drop), pulmonary (e.g., by oral or nasal inhalation), buccal, sublingual, rectal and vaginal). In certain embodiments, pipendoxifene is administered orally (e.g., as a capsule or tablet, optionally with an enteric coating). In some embodiments, pipendoxifene is administered parenterally (e.g., intravenously, subcutaneously or intradermally). In some embodiments, pipendoxifene is administered topically (e.g., dermally/epicutaneously, transdermally, mucosally, transmucosally, buccally or sublingually).

In some embodiments, pipendoxifene is administered without food. In some embodiments, pipendoxifene is administered at least about 1 or 2 hours before or after a meal. In certain embodiments, pipendoxifene is administered at least about 2 hours after an evening meal. Pipendoxifene can also be taken substantially concurrently with food (e.g., within about 0.5, 1 or 2 hours before or after a meal, or with a meal).

The composition can be administered to the subject once, twice, or three times a day. The composition can be administered to the subject once every day, every two days, or every three days. The composition can be administered to the subject over the course of at least two weeks, at least three weeks, at least four weeks, or at least five weeks. The therapeutically effective amount and the frequency of administration of, and the length of treatment with, a disclosed composition may depend on various factors, including the nature and the severity of the inflammation and/or infection/disease, the potency of the compound (e.g., pipendoxifene), the mode of administration, the age, the body weight, the general health, the gender and the diet of the subject, and the response of the subject to the treatment, and can be determined by the treating physician. In some embodiments, a disclosed compound (e.g., pipendoxifene or a pharmaceutically acceptable salt, ester, solvate, stereoisomer, tautomer, or prodrug thereof) is administered under a chronic dosing regimen. In certain embodiments, a therapeutically effective amount of a disclosed compound (e.g., pipendoxifene) is administered over a period of at least about 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).

A composition of the disclosure can also be used prophylactically to for preventing, delaying the onset of, or treating an infection or a disease or inflammation caused by a DNA virus. The prophylactically effective amount of a disclosed compound (e.g., pipendoxifene) can be any therapeutically effective amount of pipendoxifene.

In some embodiments, the composition is administered at a dose from about 125 mg/kg to about 375 mg/kg of pipendoxifene, optionally at a dose of 125 mg/kg, 250 mg/kg, or 375 mg/kg of pipendoxifene. In further embodiments, the dosage can be 0.1 to 125 mg/kg, 1-125 mg/kg, 1-100 mg/kg, 1-50 mg/kg, 1 mg/kg, 10 mg/kg, 20 mg/kg, 25 mg/kg, 30 mg/kg, 40 mg/kg, 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, or about 125 mg/kg. In some embodiments, the dose (e.g., of pipendoxifene) can be about 0.01-500 mg/kg, from about 0.1-500 mg/kg, from about 1-500 mg/kg, from about 1-250 mg/kg, about 1-200 mg/kg, about 1-150 mg/kg, about 1-100 mg/kg, about 1-50 mg/kg, about 1-25 mg/kg, about 1-20 mg/kg, about 1-15 mg/kg, about 1-10 mg/kg, about 1-5 mg/kg, about 0.1-20 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 210 mg/kg, about 220 mg/kg, about 230 mg/kg, about 240 mg/kg, about 250 mg/kg, about 260 mg/kg, about 270 mg/kg, about 280 mg/kg, about 290 mg/kg, about 300 mg/kg, about 310 mg/kg, about 320 mg/kg, about 330 mg/kg, about 340 mg/kg, about 350 mg/kg, about 360 mg/kg, about 370 mg/kg, about 380 mg/kg, about 390 mg/kg, about 400 mg/kg, about 410 mg/kg, about 420 mg/kg, about 430 mg/kg, about 440 mg/kg, about 450 mg/kg, about 460 mg/kg, about 470 mg/kg, about 480 mg/kg, about 490 mg/kg, or about 500 mg/kg. In some embodiments, the dose (e.g., of pipendoxifene) can be about 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, 205 mg/kg, 210 mg/kg, 215 mg/kg, 220 mg/kg, 225 mg/kg, 230 mg/kg, 235 mg/kg, 240 mg/kg, 245 mg/kg, 250 mg/kg, 255 mg/kg, 260 mg/kg, 265 mg/kg, 270 mg/kg, 275 mg/kg, 280 mg/kg, 285 mg/kg, 290 mg/kg, 295 mg/kg, 300 mg/kg, 305 mg/kg, 310 mg/kg, 315 mg/kg, 320 mg/kg, 325 mg/kg, 330 mg/kg, 335 mg/kg, 340 mg/kg, 345 mg/kg, 350 mg/kg, 355 mg/kg, 360 mg/kg, 365 mg/kg, 370 mg/kg, 375 mg/kg, 380 mg/kg, 385 mg/kg, 390 mg/kg, 395 mg/kg, 400 mg/kg, 405 mg/kg, 410 mg/kg, 415 mg/kg, 420 mg/kg, 425 mg/kg, 430 mg/kg, 435 mg/kg, 440 mg/kg, 445 mg/kg, 450 mg/kg, 455 mg/kg, 460 mg/kg, 465 mg/kg, 470 mg/kg, 475 mg/kg, 480 mg/kg, 485 mg/kg, 490 mg/kg, 495 mg/kg, or 500 mg/kg.

Administrating the composition can result in reduction of the viral titer of the DNA virus in the subject as compared to that in the subject before administration of the composition. The method can comprise determining global virus distribution in the lungs and/or blood of the subject. The method can comprise measuring the viral titer of the DNA virus in the subject before administering the composition to the subject, after administering the composition to the subject, or both. The viral titer can be lung bulk virus titer or blood viral titer.

The method can comprise measuring a neutrophil density within the lungs of the subject. Administering the composition can result in reduction of the neutrophil density within the lungs of the subject as compared to that in the subject before administration of the composition. Administering the composition can result in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of the neutrophil density within the lungs of the subject as compared to that in the subject before administration of the composition.

The method can comprise measuring a total necrotized cell count within the lungs of the subject. Administering the composition can result in reduction of the total necrotized cell count in the subject as compared to that in the subject before administration of the composition. The method can comprise measuring a total protein level within the lungs of the subject. Administering the composition can result in reduction of the total protein level within the lungs of the subject as compared to that in the subject before administration of the composition. In some embodiments, administering the composition results in an at least, or at least about, 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) reduction of the total protein level within the lungs of the subject as compared to that in the subject before administration of the composition.

In some embodiments, the method can comprise measuring liver enzyme levels (e.g., alanine aminotransferase (ALT) and aspartate aminotransferase (AST)) in the blood of the subject. In some embodiments, the method can comprise measuring liver enzymes prior to and after the administration of the composition. The compositions and methods provided herein can reduce the liver enzyme level by at least, or at least about 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used.

In some embodiments, the method can comprise measuring albumin level in the blood of the subject. In some embodiments, the method can comprise measuring the albumin level prior to and after the administration of the composition. The compositions and methods provided herein can increase the albumin level by at least, or at least about 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used.

The method can comprise measuring HBV DNA and/or anti-HBV antibody in the blood of the subject. In some embodiments, administering the composition results in reduction of the HBV DNA and/or anti-HBV antibody in the subject as compared to that in the subject before administration of the composition. The method can comprise measuring EBV DNA and/or anti-EBV antibody in the blood of the subject. In some embodiments, administering the composition results in reduction of the EBV DNA and/or anti-EBV antibody in the subject as compared to that in the subject before administration of the composition.

The compositions and methods provided herein can reduce the HBV or EBV RNA and/or anti-HBV or anti-EBV antibody level by at least, or at least about 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, 150%, 200%, 250%, 500%, 1000%, or higher and overlapping ranges therein) compared to if the methods and compositions are not used.

Pipendoxifene, disclosed herein, in some embodiments, have low or no binding affinity to estrogen receptor. For example, the growth inhibition IC50 of pipendoxifene to estrogen receptor can be greater than 0.1 μM, 0.5 μM, 1 μM, 3 μM, 5 μM, 10 μM, 12 μM, 13 μM, 14 μM, 15 μM, 16 μM, 17 μM, 18 μM, 19 μM, 20 μM, 50 μM, or a value or a range between any two of these values.

In some embodiments, the compounds disclosed herein (e.g., pipendoxifene) are designed to exclude undesired properties, specifically estrogen receptor interaction. In some embodiments, the compounds disclosed herein (e.g., pipendoxifene) demonstrate low estrogen receptor binding probability and negligible inhibition of estrogen receptor-mediated growth. In some embodiments, the estrogen receptor binding probability of pipendoxifene is reduced by at least, or at least about 50%, 75%, 100%, 150%, 200%, 250%, 500%, 1000%, or higher and overlapping ranges therein, compared to other antiviral compounds.

### Co-Infection

In some embodiments, the subject is co-infected with a DNA virus and another virus, e.g., an RNA virus or a DNA virus.

In some embodiments, the composition is capable of inhibiting RdRp of an RNA virus. The RNA virus can be any RNA virus encoding an RNA-dependent RNA polymerase (RdRp). In some embodiments, the RNA virus can be a double-stranded RNA virus. The RNA virus can be a positive-sense single-stranded RNA virus. The positive-sense single-stranded RNA virus can be a coronavirus. The coronavirus can be an alpha coronavirus, a beta coronavirus, a gamma coronavirus, or a delta coronavirus. The coronavirus can be human coronavirus 229E (HCoV-229E), human coronavirus NL63 (HCoV-NL63), human coronavirus OC43 (HCoV-OC43), human coronavirus HKU1 (HCoV-HKU1), Middle East respiratory coronavirus (MERS-CoV), severe acute respiratory syndrome coronavirus (SARS-COV), or SARS-COV-2. In some embodiments, the positive-sense single-stranded RNA virus is hepatitis C virus. The infection or a disease caused by the RNA virus can be common cold, influenza, SARS, coronaviruses, COVID-19, hepatitis C, hepatitis E, West Nile fever, Ebola virus disease, rabies, polio, or measles. In some embodiments, the subject is co-infected with HCV and the DNA virus. In some embodiments, the subject is co-infected with HIV and the DNA virus. In some embodiments, the subject is co-infected with HDV and the DNA virus.

Also disclosed herein are methods for preventing, delaying the onset of, or treating a HBV and HDV co-infection or a disease caused by a HBV and HDV co-infection, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the HBV and HDV co-infection or the disease.

In some embodiments, there is also provided methods for preventing, delaying the onset of, or treating an inflammatory effect of a HBV and HDV co-infection or a disease caused by a HBV and HDV co-infection, comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the inflammatory effect.

Hepatitis D is a type of viral hepatitis caused by the HDV. HDV is one of five known hepatitis viruses: A, B, C, D, and E. HDV is considered to be a satellite (a type of subviral agent) because it can propagate only in the presence of the HBV. Transmission of HDV can occur either via simultaneous infection with HBV (coinfection) or superimposed on chronic hepatitis B or hepatitis B carrier state (superinfection).

HDV infecting a person with chronic hepatitis B (superinfection) is considered the most serious type of viral hepatitis due to its severity of complications. These complications include a greater likelihood of experiencing liver failure in acute infections and a rapid progression to liver cirrhosis, with an increased risk of developing liver cancer in chronic infections. In some embodiments, the inflammatory effect comprises acute hepatitis, chronic hepatitis, liver swelling, liver damage, joint inflammation, muscle pain and weakness, or blood vessel problem. In some embodiments, the disease caused by the HBV and HDV co-infection is a liver disease. The liver disease can comprise cirrhosis and/or liver failure. In some embodiments, the disease caused by the HBV and HDV co-infection is liver cancer. In some embodiments, the subject in need thereof is a subject that is suffering from the HBV and HDV co-infection or the disease, or a subject that is at a risk for the HBV and HDV co-infection or the disease. In some embodiments, the HBV and HDV co-infection or the disease is in the liver of the subject. In some embodiments, the subject is a subject that has been exposed to HBV and/or HDV, is suspected to have been exposed to HBV and/or HDV, or is at a risk of being exposed to HBV and/or HDV. In some embodiments, the subject has or is at risk of having acute HDV and/or chronic HDV. In some embodiments, the subject is a mammal. In some embodiments, the subject is a human.

The method can comprise administering to the subject one or more additional antiviral agents. In some embodiments, at least one of the one or more additional antiviral agents is co-administered to the subject with the composition. In some embodiments, at least one of the one or more additional antiviral agents is administered to the subject before the administration of the composition, after the administration of the composition, or both. In some embodiments, the composition comprises one or more additional therapeutic agents. In some embodiments, the one or more additional therapeutic agents comprise one or more additional antiviral agents. In some embodiments, the one or more additional antiviral agents is selected from the group consisting of a nucleoside, a non-nucleoside analogue reverse-transcriptase inhibitor, a nucleotide analogue reverse-transcriptase inhibitor, an interferon and/or a sodium/bile acid cotransporter inhibitor. In some embodiments, the composition is administered to the subject by intravenous administration, nasal administration, pulmonary administration, oral administration, parenteral administration, or nebulization. In some embodiments, the composition is in the form of powder, pill, tablet, microtablet, pellet, micropellet, capsule, capsule containing microtablets, liquid, aerosols, or nanoparticles. In some embodiments, the composition is in a formulation for oral or intravenous administration. In some embodiments, the composition is administered to the subject once, twice, or three times a day. In some embodiments, the composition is administered to the subject once every day, every two days, or every three days. In some embodiments, the composition is administered to the subject over the course of at least two weeks, at least three weeks, at least four weeks, or at least five weeks. In some embodiments, the composition is administered at a dose from about 125 mg/kg to about 375 mg/kg of pipendoxifene. In some embodiments, the composition is administered at a dose of 125 mg/kg, 250 mg/kg, or 375 mg/kg of pipendoxifene.

The method can comprise measuring the viral titer of HBV and/or HDV in the subject before administering the composition to the subject, after administering the composition to the subject, or both. In some embodiments, the viral titer is blood viral titer. In some embodiments, administrating the composition results in reduction of the viral titer of the HBV and/or HDV in the subject as compared to that in the subject before administration of the composition. The method can comprise determining global HBV and/or HDV distribution in an organ or a tissue of the subject; and optionally the method comprises determining global virus distribution in the blood of the subject. The method can comprise measuring liver enzyme levels in the blood of the subject. The liver enzymes can comprise alanine aminotransferase (ALT) and aspartate aminotransferase (AST). In some embodiments, administering the composition results in reduction of the liver enzyme levels in the blood of the subject as compared to that in the subject before administration of the composition. The method can comprise measuring albumin level in the blood of the subject. In some embodiments, administering the composition results in increase of the albumin level in the blood of the subject as compared to that in the subject before administration of the composition. The method can comprise measuring HBV and/or HDV DNA in the blood of the subject. In some embodiments, administering the composition results in reduction of the HBV and/or HDV DNA in the subject as compared to that in the subject before administration of the composition. The method can comprise measuring HBV and/or HDV antibody in the blood of the subject, optionally administering the composition results in reduction of the HBV and/or HDV antibody in the subject as compared to that in the subject before administration of the composition.

### Kits and Compositions

Disclosed herein include kits. In some embodiments, the kit comprises pipendoxifene or a pharmaceutically acceptable salt, ester, solvate, stereoisomer, tautomer, or prodrug thereof; and a label indicating that the kit is for preventing, delaying the onset of, or treating an infection or a disease caused by a DNA virus; or a label indicating that the kit is for preventing, delaying the onset of, or treating an inflammatory effect of an infection or a disease caused by a DNA virus. The DNA virus can be a Baltimore class VII (BCVII) virus, e.g., a hepadnavirus and/or a caulimovirus. The DNA virus can be a pararetrovirus. The DNA virus can be HBV. The DNA virus can be a Baltimore class I (BCI) virus, e.g., Duplodnaviria, Monodnaviria, Singelaviria, or Varidnaviria. The DNA virus can be Epstein-Barr virus (EBV).

Disclosed herein include compositions for use in preventing, delaying the onset of, or treating an infection or a disease caused by a DNA virus. In some embodiments, the composition comprises pipendoxifene or a pharmaceutically acceptable salt, ester, solvate, stereoisomer, tautomer, or prodrug thereof, thereby preventing, delaying the onset of, or treating the infection or the disease.

Disclosed herein include compositions for use in preventing, delaying the onset of, or treating an inflammatory effect of an infection or a disease caused by a DNA virus. In some embodiments, the composition comprises pipendoxifene or a pharmaceutically acceptable salt, ester, solvate, stereoisomer, tautomer, or prodrug thereof, thereby preventing, delaying the onset of, or treating the infection or the disease.

Disclosed herein are compositions for use in preventing, delaying the onset of, or treating a HBV and HDV co-infection or a disease caused by a HBV and HDV co-infection, wherein the composition comprises pipendoxifene or a pharmaceutically acceptable salt, ester, solvate, stereoisomer, tautomer, or prodrug thereof, thereby preventing, delaying the onset of, or treating the HBV and HDV co-infection or the disease.

Also disclosed herein are compositions for use in preventing, delaying the onset of, or treating an inflammatory effect of a HBV and HDV co-infection or a disease caused by a HBV and HDV co-infection or a disease caused by a HBV and HDV co-infection, wherein the composition comprises pipendoxifene or a pharmaceutically acceptable salt, ester, solvate, stereoisomer, tautomer, or prodrug thereof, thereby preventing, delaying the onset of, or treating the HBV and HDV co-infection or the disease.

The inflammatory effect can comprise acute hepatitis, chronic hepatitis, liver swelling, liver damage, joint inflammation, muscle pain and weakness, and/or blood vessel problem. The inflammatory effect can comprise peritonsillar abscesses, acute bacterial sinusitis, suppurative lymph nodes, mastoiditis, and/or sialadenitis. The inflammatory effect can comprise respiratory failure, a sequela of respiratory failure, acute lung injury, and/or acute respiratory distress syndrome. The sequela of respiratory failure can comprise multi-organ failure. The composition can comprise a therapeutically or prophylactically effective amount of pipendoxifene, or a pharmaceutically acceptable salt, ester, solvate, stereoisomer, tautomer, or prodrug thereof.

The therapeutically effective amount and the frequency of administration of, and the length of treatment with, pipendoxifene or a pharmaceutically acceptable salt, ester, solvate, stereoisomer, tautomer, or prodrug thereof may depend on various factors, including the nature and the severity of the inflammation and/or infection/disease, the mode of administration, the age, the body weight, the general health, the gender and the diet of the subject, and the response of the subject to the treatment, and can be determined by the treating physician. In some embodiments, a therapeutically effective amount of pipendoxifene or a pharmaceutically acceptable salt, ester, solvate, stereoisomer, tautomer, or prodrug thereof for treating or preventing inflammation, an infection, and/or a disease as described herein is about 0.1-200 mg, 0.1-150 mg, 0.1-100 mg, 0.1-50 mg, 0.1-30 mg, 0.5-20 mg, 0.5-10 mg or 1-10 mg (e.g., per day or per dose), or as deemed appropriate by the treating physician, which can be administered in a single dose or in divided doses. In certain embodiments, the therapeutically effective dose (e.g., per day or per dose) of pipendoxifene or a pharmaceutically acceptable salt, ester, solvate, stereoisomer, tautomer, or prodrug thereof for treating or preventing inflammation, an infection, and/or a disease as described herein is about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), about 150-200 mg (e.g., about 150 mg, 175 mg or 200 mg), about 200-300 mg (e.g., about 200 mg, 220 mg, 240 mg, 260 mg, 280 mg, or 300 mg), about 300-400 mg (e.g., about 300 mg, 320 mg, 340 mg, 360 mg, 380 mg, or 400 mg), about 400-500 mg (e.g., about 400 mg, 420 mg, 440 mg, 460 mg, 480 mg, or 500 mg), about 500-600 mg (e.g., about 500 mg, 520 mg, 540 mg, 560 mg, 580 mg, or 600 mg), or about 600-700 mg (e.g., about 600 mg, 620 mg, 640 mg, 660 mg, 680 mg, or 700 mg). In some embodiments, pipendoxifene or a pharmaceutically acceptable salt, ester, solvate, stereoisomer, tautomer, or prodrug thereof is administered for treating or preventing inflammation, an infection, and/or a disease as described herein at a daily dose, weekly dose, and/or monthly dose of about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), about 150-200 mg (e.g., about 150 mg, 175 mg or 200 mg), about 200-300 mg (e.g., about 200 mg, 220 mg, 240 mg, 260 mg, 280 mg, or 300 mg), about 300-400 mg (e.g., about 300 mg, 320 mg, 340 mg, 360 mg, 380 mg, or 400 mg), about 400-500 mg (e.g., about 400 mg, 420 mg, 440 mg, 460 mg, 480 mg, or 500 mg), about 500-600 mg (e.g., about 500 mg, 520 mg, 540 mg, 560 mg, 580 mg, or 600 mg), or about 600-700 mg (e.g., about 600 mg, 620 mg, 640 mg, 660 mg, 680 mg, or 700 mg). The daily dose, weekly dose, and/or monthly dose of pipendoxifene can comprise a single administration (e.g., a weekly dose can administered once per week) or multiple administrations. In some embodiments, the dosing regimen comprises administering one or more loading doses and one or more maintenance doses. The term “loading dose” shall be given its ordinary meaning, and shall also refer to a single dose or short duration regimen of a multiple doses having a dosage higher than one or more maintenance doses. A loading dose can, for example, rapidly increase the blood concentration level of disclosed compounds. In some embodiments, the loading dose can increase the blood concentration of pipendoxifene to a therapeutically effective level in conjunction with a maintenance dose of pipendoxifene. The loading dose can be administered once per day, or more than once per day (e.g., up to 4 times per day). The term “maintenance dose” as used herein shall be given its ordinary meaning, and shall also refer to a dose that is serially administered (e.g., at least twice) which is intended to either slowly raise blood concentration levels of a disclosed compound (e.g., pipendoxifene) to a therapeutically effective level, or to maintain such a therapeutically effective level. The daily dose of the maintenance dose can lower than the total daily dose of the loading dose.

The composition can be a pharmaceutical composition comprising the compound (e.g., pipendoxifene) and one or more pharmaceutically acceptable excipients. The composition can comprise one or more additional therapeutic agents. The one or more additional therapeutic agents comprise one or more antiviral agents. The one or more antiviral agents can be selected from the group consisting of a nucleoside or a non-nucleoside analogue reverse-transcriptase inhibitor, a nucleotide analogue reverse-transcriptase inhibitor, a NS3/4A serine protease inhibitor, a NS5B polymerase inhibitor, and interferon alpha.

The composition can be in the form of powder, pill, tablet, microtablet, pellet, micropellet, capsule, capsule containing microtablets, liquid, aerosols, or nanoparticles. The composition can be in a formulation for intravenous administration. As disclosed herein, pipendoxifene can be formulated for administration in a pharmaceutical composition comprising a physiologically acceptable surface active agents, carriers, diluents, excipients, smoothing agents, suspension agents, film forming substances, coating assistants, or a combination thereof. In some embodiments, pipendoxifene or a pharmaceutically acceptable salt, ester, solvate, stereoisomer, tautomer, or prodrug thereof is formulated for administration with a pharmaceutically acceptable carrier or diluent. The pipendoxifene or a pharmaceutically acceptable salt, ester, solvate, stereoisomer, tautomer, or prodrug thereof can be formulated as a medicament with a standard pharmaceutically acceptable carrier(s) and/or excipient(s) as is routine in the pharmaceutical art. The exact nature of the formulation will depend upon several factors including the desired route of administration. Pipendoxifene (or a pharmaceutically acceptable salt, ester, solvate, stereoisomer, tautomer, or prodrug thereof) can be formulated for oral, intravenous, intragastric, intravascular or intraperitoneal administration.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose: starches, such as corn starch and potato starch; cellulose and its derivatives, such as sodium carboxymethyl cellulose, powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerin, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject therapeutic agent is basically determined by the way the composition is to be administered.

The compositions can be provided in unit dosage form. As used herein, a “unit dosage form” is a composition containing an amount of pipendoxifene or a pharmaceutically acceptable salt, ester, solvate, stereoisomer, tautomer, or prodrug thereof that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan can recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compounds of the disclosure (e.g., pipendoxifene a pharmaceutically acceptable salt, ester, solvate, stereoisomer, tautomer, or prodrug thereof). The amount of carrier employed in conjunction with the compounds of the disclosure (e.g., pipendoxifene or a pharmaceutically acceptable salt, ester, solvate, stereoisomer, tautomer, or prodrug thereof) is sufficient to provide a practical quantity of material for administration per unit dose of the disclosed compositions.

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the subject therapeutic agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

For topical use, creams, ointments, gels, solutions or suspensions, etc., containing pipendoxifene or a pharmaceutically acceptable salt, ester, solvate, stereoisomer, tautomer, or prodrug thereof disclosed herein are employed. Topical formulations may generally be comprised of a pharmaceutical carrier, co-solvent, emulsifier, penetration enhancer, preservative system, and emollient.

For intravenous administration, pipendoxifene or a pharmaceutically acceptable salt, ester, solvate, stereoisomer, tautomer, or prodrug thereof and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, suifoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Antimicrobial agents may also be included to achieve a bacteriostatic or fungistatic solution, including but not limited to phenyl mercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of pipendoxifene or a pharmaceutically acceptable salt, ester, solvate, stereoisomer, tautomer, or prodrug thereof and another agent, the combination may be provided to caregivers as a mixture, or the caregivers may mix the two agents prior to administration, or the two agents may be administered separately.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 0.1 mg/kg and 4000 mg/kg body weight, preferably between about 80 mg/kg and 1600 mg/kg body weight. Alternatively, dosages may be based and calculated upon the surface area of the patient, as understood by those of skill in the art.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will depend on many factors including the subject being treated, the severity of the affliction, the manner of administration, the judgment of the prescribing physician. The compounds of the disclosure (e.g., pipendoxifene or a pharmaceutically acceptable salt, ester, solvate, stereoisomer, tautomer, or prodrug thereof) or combination of therapeutic agents disclosed herein may be administered orally or via injection at a dose from 0.1 mg/kg to 4000 mg/kg of the patient's body weight per day. The dose range for adult humans is generally from 1 g to 100 g/day. Tablets or other forms of presentation provided in discrete units may conveniently contain an amount of pipendoxifene or a pharmaceutically acceptable salt, ester, solvate, stereoisomer, tautomer, or prodrug thereof or combination of other agents which is effective at such dosage or as a multiple of the same, for instance, units containing 1 g to 60 g (for example, from about 5 g to 20 g, from about 10 g to 50 g, from about 20 g to 40 g, or from about 25 g to 35 g). The precise amount of therapeutic agent administered to a patient is the responsibility of the attendant physician. However, the dose employed can depend on a number of factors, including the age and sex of the patient, the precise disorder being treated, and its severity. Additionally, the route of administration may vary depending on the condition and its severity. A typical dose of pipendoxifene or a pharmaceutically acceptable salt, ester, solvate, stereoisomer, tautomer, or prodrug thereof can be from 0.02 g to 1.25 g per kg of body weight, for example from 0.1 g to 0.5 g per kg of body weight, depending on such parameters. In some embodiments, the dosage can be from 1 g to 100 g, for example, from 10 g to 80 g, from 15 g to 60 g, from 20 g to 40 g, or from 25 g to 35 g. In A physician will be able to determine the required dosage for any particular subject.

The exact formulation, route of administration and dosage for the pharmaceutical compositions comprising pipendoxifene (or a pharmaceutically acceptable salt, ester, solvate, stereoisomer, tautomer, or prodrug thereof) or combination of therapeutic agents disclosed herein can be chosen by the individual physician in view of the patient's condition. (See, e.g., Fingl et al. 1975, in “The Pharmacological Basis of Therapeutics,” which is hereby incorporated herein by reference, with particular reference to Ch. 1). Typically, the dose range of the composition administered to the patient can be from about 0.1 to about 4000 mg/kg of the patient's body weight. The dosage may be a single one or a series of two or more given in the course of one or more days, as is needed by the patient. In instances where human dosages for therapeutic agents have been established for at least some condition, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. In cases of administration of a pharmaceutically acceptable salt, dosages may be calculated as the free base. In some embodiments, the composition is administered 1 to 4 times per day. Alternatively the compositions disclosed herein may be administered by continuous intravenous infusion, e.g., at a dose of each active ingredient up to 100 g per day. As will be understood by those of skill in the art, in certain situations it may be necessary to administer the compositions disclosed herein in amounts that exceed, or even far exceed, the above-stated, preferred dosage range in order to effectively and aggressively treat particularly aggressive diseases or infections. In some embodiments, a compound of the disclosure (e.g., pipendoxifene or a pharmaceutically acceptable salt, ester, solvate, stereoisomer, tautomer, or prodrug thereof) or combination of therapeutic agents disclosed herein will be administered for a period of continuous therapy, for example for a week or more, or for months or years.

In some embodiments, the dosing regimen of pipendoxifene or a pharmaceutically acceptable salt, ester, solvate, stereoisomer, tautomer, or prodrug thereof or combination of therapeutic agents disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of pipendoxifene or a pharmaceutically acceptable salt, ester, solvate, stereoisomer, tautomer, or prodrug thereof or combination of therapeutic agents disclosed herein can be administered three times a day, twice a day, daily, every other day, three times a week, every other week, three times per month, once monthly, substantially continuously or continuously.

In some embodiments, a pharmaceutical composition comprises pipendoxifene or a pharmaceutically acceptable salt, solvate, hydrate, clathrate, polymorph, tautomer, prodrug or metabolite thereof, and one or more pharmaceutically acceptable carriers or excipients. The composition can optionally contain one or more additional therapeutic agents as described herein. A pharmaceutical composition can comprise a therapeutically effective amount of pipendoxifene, or a pharmaceutically acceptable salt, ester, solvate, stereoisomer, tautomer, or prodrug thereof and one or more pharmaceutically acceptable carriers or excipients, and is formulated for administration to a subject for therapeutic use. For purposes of the content of a pharmaceutical composition, the terms “therapeutic agent”, “active ingredient”, “active agent” and “drug” encompass prodrugs.

A pharmaceutical composition can contain pipendoxifene or a pharmaceutically acceptable salt, ester, solvate, stereoisomer, tautomer, or prodrug thereof in substantially pure form. In some embodiments, the purity of the therapeutic agent is at least about 95%, 96%, 97%, 98% or 99%. In certain embodiments, the purity of the therapeutic agent is at least about 98% or 99%. In addition, a pharmaceutical composition can be substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501 (a) (2) (B) and the International Conference on Harmonisation Q7 Guideline.

Pharmaceutically acceptable carriers and excipients include pharmaceutically acceptable materials, vehicles and substances. Non-limiting examples of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, stabilizers, preservatives, antioxidants, antimicrobial agents, antibacterial agents, antifungal agents, absorption-delaying agents, sweetening agents, flavoring agents, coloring agents, adjuvants, encapsulating materials and coating materials. The use of such excipients in pharmaceutical formulations is known in the art. For example, conventional vehicles and carriers include without limitation oils (e.g., vegetable oils, such as sesame oil), aqueous solvents (e.g., saline, phosphate-buffered saline (PBS) and isotonic solutions [e.g., Ringer's solution]), and solvents (e.g., dimethyl sulfoxide (DMSO) and alcohols (e.g., ethanol, glycerol and propylene glycol)). Except insofar as any conventional carrier or excipient is incompatible with the active ingredient, the disclosure encompasses the use of conventional carriers and excipients in formulations containing a therapeutic agent.

Proper formulation can depend on various factors, such as the mode of administration chosen. Potential modes of administration of pharmaceutical compositions include without limitation oral, parenteral (including intramuscular, subcutaneous, intradermal, intravascular, intravenous, intra-arterial, intraperitoneal, intramedullary, intrathecal and topical), intra-cavitary, and topical (including dermal/epicutaneous, transdermal, mucosal, transmucosal, intranasal (e.g., by nasal spray or drop), pulmonary (e.g., by oral or nasal inhalation), buccal, sublingual, rectal (e.g., by suppository), and vaginal (e.g., by suppository).

As an example, formulations of the disclosure suitable for oral administration can be presented as, e.g., boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Tablets can contain pipendoxifene or a pharmaceutically acceptable salt, ester, solvate, stereoisomer, tautomer, or prodrug thereof in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In certain embodiments, a tablet comprises pipendoxifene (or a pharmaceutically acceptable salt, ester, solvate, stereoisomer, tautomer, or prodrug thereof), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can comprise pipendoxifene or a pharmaceutically acceptable salt, ester, solvate, stereoisomer, tautomer, or prodrug thereof in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, a compound of the disclosure (e.g., pipendoxifene or a pharmaceutically acceptable salt, ester, solvate, stereoisomer, tautomer, or prodrug thereof) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules pipendoxifene or a pharmaceutically acceptable salt, ester, solvate, stereoisomer, tautomer, or prodrug thereof can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

In some embodiments, pipendoxifene (or a pharmaceutically acceptable salt, ester, solvate, stereoisomer, tautomer, or prodrug thereof) is contained in an amphiphilic vehicle of a liquid or semi-solid formulation for oral administration which provides improved solubility, stability and bioavailability of the compound, as described in US 2010/0209496. The amphiphilic vehicle contains a solution, suspension, emulsion (e.g., oil-in-water emulsion) or semi-solid mixture of the compound admixed with liquid or/and semi-solid excipients which fills an encapsulated dosage form (e.g., a hard gelatin capsule or a soft gelatin capsule containing a plasticizer (e.g., glycerol or/and sorbitol)). In some embodiments, the amphiphilic vehicle comprises an amphiphilic agent selected from fatty acid esters of glycerol (glycerin), propylene glycol and sorbitol. In certain embodiments, the amphiphilic agent is selected from mono- and di-glycerides of C₈-C₁₂ saturated fatty acids. In further embodiments, the amphiphilic agent is selected from CAPMUL® MCM, CAPMUL® MCM 8, CAPMUL® MCM 10, IMWITOR® 308, IMWITOR® 624, IMWITOR® 742, IMWITOR® 988, CAPRYOL™ PGMC, CAPRYOL™ 90, LAUROGLYCOL™ 90, CAPTEX® 200, CRILL™ 1, CRILL™ 4, PECEOL® and MAIS INE™ 35-1. In some embodiments, the amphiphilic vehicle further comprises propylene glycol, a propylene glycol-sparing agent (e.g., ethanol or/and glycerol), or an antioxidant (e.g., butylated hydroxyanisole, butylated hydroxytoluene, propyl gallate or/and sodium sulfite), or any combination thereof. In additional embodiments, the amphiphilic vehicle contains on a weight basis about 0.1-5% of the compound, about 50-90% of the amphiphilic agent, about 5-40% of propylene glycol, about 5-20% of the propylene glycol-sparing agent, and about 0.01-0.5% of the antioxidant.

Pipendoxifene (or a pharmaceutically acceptable salt, ester, solvate, stereoisomer, tautomer, or prodrug thereof) can also be formulated for parenteral administration by injection or infusion to circumvent gastrointestinal absorption and first-pass metabolism. A representative parenteral route is intravenous.

Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain a compound of the disclosure (e.g., pipendoxifene or a pharmaceutically acceptable salt, ester, solvate, stereoisomer, tautomer, or prodrug thereof) along with excipients such as an antioxidant, a buffer, a bacteriostat and solutes that render the formulation isotonic with the blood of the subject. Aqueous or non-aqueous sterile suspensions can contain pipendoxifene or a pharmaceutically acceptable salt, ester, solvate, stereoisomer, tautomer, or prodrug thereof along with excipients such as a suspending agent and a thickening agent, and optionally a stabilizer and an agent that increases the solubility of the compound to allow for the preparation of a more concentrated solution or suspension. As another example, a sterile aqueous solution for injection or infusion (e.g., subcutaneously or intravenously) can contain pipendoxifene or a pharmaceutically acceptable salt, ester, solvate, stereoisomer, tautomer, or prodrug thereof, NaCl, a buffering agent (e.g., sodium citrate), a preservative (e.g., meta-cresol), and optionally a base (e.g., NaOH) or/and an acid (e.g., HCl) to adjust pH.

For topical administration, pipendoxifene or a pharmaceutically acceptable salt, ester, solvate, stereoisomer, tautomer, or prodrug thereof can be formulated as, e.g., a buccal or sublingual tablet or pill. Advantages of a buccal or sublingual tablet or pill include avoidance of first-pass metabolism and circumvention of gastrointestinal absorption. A buccal or sublingual tablet or pill can also be designed to provide faster release of the compound for more rapid uptake of it into systemic circulation. In addition to a therapeutically effective amount of pipendoxifene or a pharmaceutically acceptable salt, ester, solvate, stereoisomer, tautomer, or prodrug thereof, the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, pipendoxifene or a pharmaceutically acceptable salt, ester, solvate, stereoisomer, tautomer, or prodrug thereof can also be formulated for intranasal administration. The nasal mucosa provides a big surface area, a porous endothelium, a highly vascular subepithelial layer and a high absorption rate, and hence allows for high bioavailability. An intranasal solution or suspension formulation can pipendoxifene or a pharmaceutically acceptable salt, ester, solvate, stereoisomer, tautomer, or prodrug thereof along with excipients such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. In certain embodiments, a nasal spray formulation comprises pipendoxifene or a pharmaceutically acceptable salt, ester, solvate, stereoisomer, tautomer, or thereof, microcrystalline cellulose, sodium prodrug carboxymethylcellulose, dextrose and water, and optionally an acid (e.g., HCl) to adjust pH. An intranasal solution or suspension formulation can be administered to the nasal cavity by any suitable means, including but not limited to a dropper, a pipette, or spray using, e.g., a metering atomizing spray pump.

An additional mode of topical administration is pulmonary, including by oral inhalation and nasal inhalation, which is described in detail below. Other suitable topical formulations and dosage forms include without limitation ointments, creams, gels, lotions, pastes and the like.

Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g., menthol), opacifiers, fragrances and colorants. For an active agent having a low rate of permeation through the skin or mucosal tissue, a topical formulation can contain a permeation enhancer to increase the permeation of the active agent through the skin or mucosal tissue. A topical formulation can also contain an irritation-mitigating excipient that reduces any irritation to the skin or mucosa caused by the active agent, the permeation enhancer or any other component of the formulation.

In some embodiments pipendoxifene or a pharmaceutically acceptable salt, ester, solvate, stereoisomer, tautomer, or prodrug thereof is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug or an active metabolite thereof delivered to the target site(s) over a time period, including delivery of a therapeutically effective amount of the drug or an active metabolite thereof over a prolonged time period. In certain embodiments, the sustained-release composition delivers the compound over a period of at least about 1 day, 2 days, 3 days, 1 week, 2 weeks, 3 weeks, 1 month, 2 months, 3 months or longer. In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium poly acrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

Pharmaceutical compositions comprising pipendoxifene or a pharmaceutically acceptable salt, ester, solvate, stereoisomer, tautomer, or prodrug thereof can be formulated as, e.g., liposomes, micelles (e.g., those composed of biodegradable natural or/and synthetic polymers, such as lactosomes), microspheres, microparticles or nanoparticles, whether or not designed for sustained release.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

A pharmaceutical composition can be presented in unit dosage form as a single dose wherein all active and inactive ingredients are combined in a suitable system, and components do not need to be mixed to form the composition to be administered. The unit dosage form can contain an effective dose, or an appropriate fraction thereof, of a therapeutic agent (e.g., pipendoxifene or a pharmaceutically acceptable salt, ester, solvate, stereoisomer, tautomer, or prodrug thereof). Representative examples of a unit dosage form include a tablet, capsule or pill for oral administration, and powder in a vial or ampoule for oral or nasal inhalation.

Alternatively, a pharmaceutical composition can be presented as a kit, wherein the active ingredient, excipients and carriers (e.g., solvents) are provided in two or more separate containers (e.g., ampoules, vials, tubes, bottles or syringes) and need to be combined to form the composition to be administered. The kit can contain instructions for storing, preparing and administering the composition (e.g., a solution to be injected intravenously). A kit can contain all active and inactive ingredients in unit dosage form or the active ingredient and inactive ingredients in two or more separate containers, and can contain instructions for using the pharmaceutical composition. The kit can contain pipendoxifene or a pharmaceutically acceptable salt, solvate, hydrate, clathrate, polymorph, prodrug or metabolite thereof, and instructions for administering the compound (e.g., pipendoxifene). In certain embodiments, the compound (e.g., pipendoxifene) is contained or incorporated in, or provided by, a device or system configured for pulmonary delivery of the compound by oral inhalation, such as a metered-dose inhaler, a dry powder inhaler or a nebulizer.

Pulmonary administration can be accomplished by, e.g., oral inhalation or nasal inhalation. Advantages of pulmonary drug delivery include, but are not limited to: 1) avoidance of first pass hepatic metabolism; 2) fast drug action; 3) large surface area of the alveolar region for absorption, high permeability of the lungs (thin air-blood barrier), and profuse vasculature of the airways; 4) smaller doses to achieve equivalent therapeutic effect compared to other oral routes; 5) local action within the respiratory tract; 6) reduced systemic side effects; and 7) reduced extracellular enzyme levels compared to the gastrointestinal tract due to the large alveolar surface area. An advantage of oral inhalation over nasal inhalation includes deeper penetration/deposition of the drug into the lungs. Pulmonary administration, whether by oral or nasal inhalation, can be a suitable route of administration for drugs that are intended to act locally in the lungs or/and systemically, for which the lungs serve as a portal to the systemic circulation.

Oral or nasal inhalation can be achieved by means of, e.g., a metered-dose inhaler (MDI), a nebulizer or a dry powder inhaler (DPI). For example, pipendoxifene or a pharmaceutically acceptable salt, ester, solvate, stereoisomer, tautomer, or prodrug thereof can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer. For example, an MDI formulation can comprise pipendoxifene or a pharmaceutically acceptable salt, ester, solvate, stereoisomer, tautomer, or prodrug thereof, a propellant (e.g., an HFA such as 1,1,1,2-tetrafluoroethane), a surfactant (e.g., a fatty acid such as oleic acid), and a co-solvent (e.g., an alcohol such as ethanol). The MDI formulation can optionally contain a dissolved gas (e.g., CO₂). After device actuation, the bursting of CO₂ bubbles within the emitted aerosol droplets breaks up the droplets into smaller droplets, thereby increasing the respirable fraction of drug. As another example, a nebulizer formulation can comprise pipendoxifene or a pharmaceutically acceptable salt, ester, solvate, stereoisomer, tautomer, or prodrug thereof, a surfactant (e.g., a Tween® such as polysorbate 80), a chelator or preservative (e.g., edetate disodium), an isotonicity agent (e.g., sodium chloride), pH buffering agents (e.g., citric acid/sodium citrate), and water. The drug can be delivered by means of, e.g., a nebulizer or an MDI with or without a spacer, and the drug dose delivered can be controlled by a metering chamber (nebulizer) or a metering valve (MDI).

Metered-dose inhalers (also called pressurized metered-dose inhalers (pMDI)) are the most widely used inhalation devices. A metering valve delivers a precise amount of aerosol (e.g., about 20-100 μL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easibreathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer.

Nebulizers generate aerosol droplets of about 1-5 microns. They do not require user coordination between device actuation and inhalation, which can significantly affect the amount of drug deposited in the lungs. Compared to MDIs and DPIs, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™ NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, Micro Air® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse.

Respimat® Soft Mist™ inhaler combines advantages of an MDI and a nebulizer. It is a small, hand-held inhaler that does not need a power supply (like an MDI) and slowly aerosolizes a propellant-free drug solution as a soft mist (like a nebulizer), thereby reducing drug deposition in the oropharyngeal region and increasing drug deposition in the central and peripheral lung regions. The Soft Mist™ inhaler can create a large fraction of respirable droplets with slow velocity from a metered volume of drug solution. A drug delivered from the Soft Mist™ inhaler can potentially achieve the same therapeutic outcome at a significantly lower dose compared to delivery from an MDI.

For oral or nasal inhalation using a dry powder inhaler (DPI), pipendoxifene or a pharmaceutically acceptable salt, ester, solvate, stereoisomer, tautomer, or prodrug thereof can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation, and determines drug deposition in the lungs. (While a passive DPI requires rapid inspiratory airflow to de-agglomerate drug particles, rapid inspiration is not recommended with an MDI or nebulizer, since it creates turbulent airflow and fast velocity which increase drug deposition by impaction in the upper airways.) Compared to an MDI, a DPI (including a passive, breath-activated DPI) can potentially deliver larger doses of drug, and larger-size drugs (e.g., macromolecules), to the lungs.

Dry powder inhalers can be classified by dose type into single-unit dose (including disposable and reusable) and multi-dose (including multi-dose reservoirs and multi-unit dose). In a single-unit dose DPI, the formulation can be a powder mix of a micronized drug powder and a carrier and can be supplied in individual capsules, which are inserted into the inhaler for a single dose and are removed and discarded after use. The capsule body containing the dose falls into the device, while the cap is retained in the entry port for subsequent disposal. As the user inhales, the portion of the capsule containing the drug experiences erratic motion in the airstream, causing dislodged particles to be entrained and subsequently inhaled. Particle de-aggregation is caused mainly by turbulence promoted by the grid upstream of the mouthpiece or nosepiece. Examples of single-unit dose DPIs include without limitation Aerolizer®, AIR®, Conix One® (foil seal), Diskhaler®, Diskus®, Handihaler®, Microhaler®, Rotahaler® and Turbo Spin®.

A multi-unit dose DPI uses factory-metered and -sealed doses packaged in a manner so that the device can hold multiple doses without the user having to reload. The packaging typically contains replaceable disks or cartridges, or strips of foil-polymer blister packaging that may or may not be reloadable. For example, individual doses can be packaged in blister packs on a disk cassette. Following piercing, inspiratory flow through the packaging depression containing the drug induces dispersion of the powder. The aerosol stream is mixed with a bypass flow entering through holes in the mouthpiece or nosepiece, which gives rise to turbulence and promotes particle de-agglomeration. Advantages of the prepackaging include protection from the environment until use and ensurance of adequate control of dose uniformity. Examples of multi-unit dose DPIs include without limitation Acu-Breath®, Bulkhaler®, Certihaler®, DirectHaler®, Diskhaler®, Diskus®, Dispohaler®, MR, MF-DPI®, Miat-Haler®, NEXT DPI®, Prohaler®, Swinhaler® and Technohaler®.

Disclosed herein include compositions for use in preventing, delaying the onset of, or treating an infection or a disease caused by a DNA virus. In some embodiments, the composition comprises pipendoxifene or a pharmaceutically acceptable salt, ester, solvate, stereoisomer, tautomer, or prodrug thereof, thereby preventing, delaying the onset of, or treating the infection or the disease.

Disclosed herein include compositions for use in preventing, delaying the onset of, or treating an inflammatory effect of an infection or a disease caused by a DNA virus. In some embodiments, the composition comprises pipendoxifene or a pharmaceutically acceptable salt, ester, solvate, stereoisomer, tautomer, or prodrug thereof, thereby preventing, delaying the onset of, or treating the infection or the disease.

The DNA virus can be a double-stranded DNA virus. The DNA virus can be a Baltimore class VII (BCVII) virus, e.g., a hepadnavirus and/or a caulimovirus. The DNA virus can be a pararetrovirus. The DNA virus can be hepatitis B virus (HBV). The DNA virus can be a Baltimore class I (BCI) virus, e.g., Duplodnaviria, Monodnaviria, Singelaviria, or Varidnaviria. The DNA virus can be Epstein-Barr virus (EBV). The infection or the disease caused by the DNA virus can be a liver disease, pneumonia, interstitial lung disease, pancreatitis, myocarditis, mononucleosis, cancer, systemic lupus erythematosus (SLE), Sjögren's syndrome, rheumatoid arthritis, and/or multiple sclerosis (MS). The liver disease can comprise cirrhosis and/or liver failure. The cancer can be liver cancer. The cancer can comprise Burkitt lymphoma (BL), gastric carcinoma, Hodgkin lymphoma (HL), NK/T cell lymphoma (NKTL), nasopharyngeal carcinoma (NPC), diffused large B cell lymphoma (DLBCL), HIV-associated lymphomas, and/or post-transplant lymphoproliferative disease (PTLD). The composition can be a pharmaceutical composition comprising pipendoxifene or a pharmaceutically acceptable salt, ester, solvate, stereoisomer, tautomer, or prodrug thereof, and one or more pharmaceutically acceptable excipients. The composition can comprise one or more additional therapeutic agents. The one or more additional therapeutic agents can comprise one or more antiviral agents. The one or more antiviral agents is selected from the group consisting of a nucleoside, a non-nucleoside analogue reverse-transcriptase inhibitor, a nucleotide analogue reverse-transcriptase inhibitor, and/or a sodium/bile acid cotransporter inhibitor. The composition can be in the form of powder, pill, tablet, microtablet, pellet, micropellet, capsule, capsule containing microtablets, liquid, aerosols, or nanoparticles. The composition can be in a formulation for oral or intravenous administration.

Disclosed herein are compositions for use for preventing, delaying the onset of, or treating a hepatitis B virus (HBV) and hepatitis D virus (HDV) co-infection or a disease caused by a HBV and HDV co-infection, wherein the composition comprises pipendoxifene or a pharmaceutically acceptable salt, ester, solvate, stereoisomer, tautomer, or prodrug thereof, thereby preventing, delaying the onset of, or treating the HBV and HDV co-infection or the disease.

Also disclosed herein are compositions for use in preventing, delaying the onset of, or treating an inflammatory effect of a hepatitis B virus (HBV) and hepatitis D virus (HDV) co-infection or a disease caused by a hepatitis B virus (HBV) and hepatitis D virus (HDV) co-infection or a disease caused by a HBV and HDV co-infection, wherein the composition comprises pipendoxifene or a pharmaceutically acceptable salt, ester, solvate, stereoisomer, tautomer, or prodrug thereof, thereby preventing, delaying the onset of, or treating the HBV and HDV co-infection or the disease.

The composition can comprise a therapeutically or prophylactically effective amount of pipendoxifene, or a pharmaceutically acceptable salt, ester, solvate, stereoisomer, tautomer, or prodrug thereof. In some embodiments, the disease caused by the HBV and HDV co-infection is a liver disease, optionally the liver disease comprises cirrhosis and/or liver failure. In some embodiments, the disease caused by the HBV and HDV co-infection is liver cancer. In some embodiments, the composition is a pharmaceutical composition comprising pipendoxifene or a pharmaceutically acceptable salt, ester, solvate, stereoisomer, tautomer, or prodrug thereof, and one or more pharmaceutically acceptable excipients. In some embodiments, the composition comprises one or more additional therapeutic agents, optionally the one or more additional therapeutic agents comprise one or more antiviral agents. In some embodiments, the one or more antiviral agents is selected from the group consisting of a nucleoside, a non-nucleoside analogue reverse-transcriptase inhibitor, a nucleotide analogue reverse-transcriptase inhibitor, an interferon, and/or a sodium/bile acid cotransporter inhibitor. In some embodiments, the composition is in the form of powder, pill, tablet, microtablet, pellet, micropellet, capsule, capsule containing microtablets, liquid, aerosols, or nanoparticles. In some embodiments, the composition is in a formulation for oral or intravenous administration.

### Additional Therapeutic Agents

The methods disclosed herein can comprise administering to the subject in need thereof one or more additional therapeutic agents (e.g., antiviral agents). The additional therapeutic agents (e.g., antiviral agents) can be co-administered to the subject with the composition. The additional therapeutic agents can be administered to the subject before the administration of the composition, after the administration of the composition, or both. The composition can comprise one or more additional therapeutic agents.

In some embodiments, the one or more additional antiviral agents is selected from the group consisting of a nucleoside, a non-nucleoside analogue reverse-transcriptase inhibitor, a nucleotide analogue reverse-transcriptase inhibitor, and/or a sodium/bile acid cotransporter inhibitor. In some embodiments, the one or more additional antiviral agents is selected from the group consisting of a nucleoside, a non-nucleoside analogue reverse-transcriptase inhibitor, a nucleotide analogue reverse-transcriptase inhibitor, an interferon and/or a sodium/bile acid cotransporter inhibitor.

As disclosed herein, co-administration of particular ratios and/or amounts of pipendoxifene or a pharmaceutically acceptable salt, ester, solvate, stereoisomer, tautomer, or prodrug thereof and one or more additional therapeutic agents (e.g., antiviral agents) can result in synergistic effects in preventing, delaying the onset of, or treating an infection, disease, or inflammatory effect caused by a DNA virus. These synergistic effects can be such that the one or more effects of the combination compositions are greater than the one or more effects of each component alone at a comparable dosing level, or they can be greater than the predicted sum of the effects of all of the components at a comparable dosing level, assuming that each component acts independently. The synergistic effect can be, be about, be greater than, or be greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is, for example, a reduction in liver inflammation and that liver inflammation is reduced to a greater degree as compared to the sum of the effects of administering each component, determined as if each component exerted its effect independently, also referred to as the predicted additive effect herein. For example, if a composition comprising component (a) yields an effect of a 20% reduction in liver inflammation and a composition comprising component (b) yields an effect of 50% reduction in liver inflammation, then a composition comprising both component (a) and component (b) would have a synergistic effect if the combination composition's effect on liver inflammation was greater than 70%.

A synergistic combination composition can have an effect that is greater than the predicted additive effect of administering each component of the combination composition alone as if each component exerted its effect independently. For example, if the predicted additive effect is 70%, an actual effect of 140% is 70% greater than the predicted additive effect or is 1 fold greater than the predicted additive effect. The synergistic effect can be at least, or at least about, 20, 50, 75, 90, 100, 150, 200 or 300% greater than the predicted additive effect. In some embodiments, the synergistic effect can be at least, or at least about, 0.2, 0.5, 0.9, 1.1, 1.5, 1.7, 2, or 3 fold greater than the predicted additive effect.

In some embodiments, the synergistic effect of the combination compositions can also allow for reduced dosing amounts, leading to reduced side effects to the subject and reduced cost of treatment. Furthermore, the synergistic effect can allow for results that are not achievable through any other treatments. Therefore, proper identification, specification, and use of combination compositions can allow for significant improvements in the reduction and prevention of liver inflammation.

The additional therapeutic agents can include antagonists of transient receptor potential cation channels, including but not limited to transient receptor potential ankyrin A1 (TRPA1) antagonists. The additional therapeutic agents provided herein can include TRPV1 agonists that cause decrease in TRPV1 activity (desensitization) upon prolonged exposure of TRPV1 to the stimuli, including but not limited to capsaicin, camphor, carvacrol, menthol, methyl salicylate, resiniferatoxin, tinyatoxin, and analogs, derivatives and salts thereof.

The additional therapeutic agents can include antagonists of protease-activated receptors (PARs) and inhibitors of activating proteases. The additional therapeutic agents can include antagonists of endothelin receptors, including but not limited to selective endothelin A receptor (ETAR) antagonists. The additional therapeutic agents provided herein can include inhibitors of Toll-like receptors (TLRs). The additional therapeutic agents can include inhibitors of mitogen-activated protein (MAP) kinases. The additional therapeutic agents can include inhibitors of mitogen-activated protein kinase kinases (MEKs).

The additional therapeutic agents can include inhibitors of calcitonin gene-related peptide (CGRP) or receptor therefor or the production thereof. The additional therapeutic agents can include inhibitors of gastrin-releasing peptide (GRP) or the receptor therefor (GRPR, aka bombesin receptor 2 [BBR2]) or the production thereof, including but not limited to CRPR antagonists (e.g.; RC-3095), and analogs, derivatives and salts thereof.

The additional therapeutic agents provided herein can include inhibitors of nerve growth factor (NGF) or receptors therefor tropomyosin kinase receptor A [TrkA]) or the production thereof, including but not limited to NGF inhibitors (e.g., fulranumab and tanezumab), NGF receptor inhibitors (e.g., TrkA inhibitors such as A0879, CT327 and K252a), and analogs, derivatives, fragments and salts thereof. The additional therapeutic agents provided herein can include inhibitors of neurotensin or receptors therefor (e.g., neurotensin receptor 1 [NTSR1], NTSR2 and so 1) or the production thereof, including but not limited to selective NTSR1 antagonists (e.g., SR-48,692), selective NTSR2 antagonists (e.g., levocabastine), unselective receptor antagonists (e.g., SR-142,948), and analogs, derivatives and salts thereof.

The additional therapeutic agents provided herein can include inhibitors of somatostatin or receptors therefor (e.g., somatostatin receptors [SSTRs] 1 to 5) or the production thereof, including but not limited to selective SSTR2 antagonists (e.g., CYN 154806), selective SSTRS antagonists (e.g., BIM 23056), unselective SSTR antagonists (e.g., cyclosomatostatin), and analogs, derivatives, fragments and salts thereof. The additional therapeutic agents provided herein can include inhibitors of vasoactive intestinal peptide (VIP) or receptors therefor (e.g., VIPR1 and VIPR2) or the production thereof, including but not limited to VIP receptor antagonists {e.g., PG 97-269, ViPhyb, VIP(6-28)-NH₂, [p-Cl-D-Phe⁶, Leu¹⁷]VIP-NH₂, [Ac-His¹, D-Phe², Lys¹⁵, Arg¹⁶]VIP(3-7)GRF(8-27)-NH₂, and [Ac-Tyr¹, D-Phe²]GRF (1-29)-NH₂}, and analogs, derivatives, fragments and salts thereof. The additional therapeutic agents provided herein can include inhibitors of bradykinin or receptors therefor (e.g., B1 and B2) or the production thereof, including but not limited to bradykinin inhibitors (e.g., aloe, bromelain and polyphenols), bradykinin receptor B2 antagonists (e.g., icatibant and FR-173657), inhibitors of kallikreins (e.g., ecallantide, camostat, nafamostat, gabexate and C1-inhibitor), and analogs, derivatives and salts thereof. The additional therapeutic agents provided herein can include inhibitors of corticotropin-releasing hormone (CRH, aka corticoliberin) or receptors therefor (e.g., CRHR1 and CRHR2) or the production thereof, including but not limited to CRHR1 antagonists (e.g., antalarmin, pexacerfont, CP-154,526 LWH-234, NBI-27914 and R-121,919), CRHR2 antagonists (e.g., astressin-B), and analogs, derivatives and salts thereof.

The additional therapeutic agents provided herein can include antihistamines, including but not limited to antihistamines that inhibit action at the histamine H₁ receptor (e.g., acrivastine, antazoline, azatadine, astemizole, azelastine, bepotasiine, bilastine, bromodiphenhydramine, brompheniramine, buclizine, carbinoxamine, cetirizine, chlorcyclizine, chlorodiphenhydramine, chlorpheniramine, chlorpromazine, chloropyramine, cidoxepin, clemastine, cyclizine, cyproheptadine, desloratadine, dexbrompheniramine, dexchlorpheniramine, dimenhydrinate, dimetindene, diphenhydramine, doxepin, doxylamine, ebastine, embramine, esmirtazapine [(S)-(+)-enantiomer of mirtazapine], fexofenadine, hydroxyzine, ketotifen, levocabastine, levocetirizine, loratadine, meclozine mepyramine, mirtazapine, mizolastine, olopatadine, orphenadrine, phenindamine, pheniramine, phenyltoloxamine, promethazine, pyrilamine, quetiapine, quifenadine, rupatadine, terfenadine, trimeprazine tripelennamine and triprolidine), antihistamines that inhibit action at the histamine H₃ receptor (e.g., betahistine, burimamide, ciproxifan, clobenpropit, conessine, failproxifan, impentamine, iodophenpropit, irdabisant, pitolisant, thioperamide, A-349,821, ABT-239 and VUF-568), antihistamines that inhibit action at the histamine H₄ receptor (e.g., clobenpropit, thioperamide, A943931, A987306, JNJ-7777120, VUF-6002 and ZPL-389), and analogs, derivatives and salts thereof.

The additional therapeutic agents provided herein can include inhibitors of phospholipase A2 (e.g., secreted and cytosolic PLA2), including but not limited to arachidonyl trifluoromethyl ketone, bromoenol lactone, chloroquine, cytidine 5-diphosphoamines, darapladib, quinacrine, vitamin E, RO-061606, ZPL-521, lipocortins (annexins), and analogs, derivatives, fragments and salts thereof.

The additional therapeutic agents provided herein can include inhibitors of pro-inflammatory prostaglandins (e.g., prostaglandin E2) or receptors therefor or the production thereof, including but not limited to non-steroidal anti-inflammatory drugs (NSAIDs) (e.g., non-selective COX-1/COX-2 inhibitors such as aspirin and selective COX-2 inhibitors such as coxibs), glucocorticoids, cyclopentenone prostaglandins (e.g., prostaglandin J2 [PGJ2], Δ12-PGJ2 and 15-deoxy-Δ12,14-PGJ2), and analogs, derivatives and salts thereof, inhibitors of leukotrienes or receptors therefor or the production thereof, including but not limited to leukotriene receptor antagonists (e.g., cinalukast, gemilukast, iralukast, montelukast, pranlukast, tomelukast, verlukast, zafirlukast, CP-199330, HAMI-3379, ICI-198615 and MK-571), 5-lipoxygenase inhibitors (e.g., baicalein, caffeic acid, curcumin, hyperforin, meclofenamic acid, meclofenamate sodium, zileuton and MK-886), and analogs, derivatives and salts thereof.

The additional therapeutic agents provided herein can include mast cell stabilizers, including but not limited to cromoglicic acid (cromolyn), ketotifen, methylxanthines, nedocromil, olopatadine, omalizumab, pemirolast, quercetin. The additional therapeutic agents provided herein can include β₂-adrenoreceptor agonists including short-acting β₂-adrenergic agonists (e.g., bitolterol, fenoterol, isoprenaline (isoproterenol), levosalbutamol (levalbuterol), orciprenaline (metaproterenol), pirbuterol, procaterol, ritodrine, salbutamol (albuterol) and terbutaline), long-acting β₂-adrenergic agonists (e.g., arformoterol, bambuterol, clenbuterol, formoterol and salmeterol), and ultralong-acting β₂-adrenergic agonists (e.g., carmoterol, indacaterol, milveterol, olodaterol and vilanterol), and analogs, derivatives and salts thereof.

The additional therapeutic agents can include Janus kinase (JAX) inhibitors, including, but not limited to JAK1 inhibitors (e.g., GLPG0634 and GSK2586184). JAK2 inhibitors, dual JAK1/JAK2 inhibitors (e.g., baricitinib and ruxolitinib), dual JAK1/JAK3 inhibitors (e.g., tofacitinib), and analogs, derivatives and salts thereof.

The additional therapeutic agents can include immunomodulators, including but not limited to imides (e.g., thalidomide, lenalidomide, pomalidomide and apremilast), xanthine derivatives (e.g., lisofylline, pentoxifylline and propentofylline), and analogs, derivatives and salts thereof. The additional therapeutic agents can include immunosuppressants, including but not limited to glucocorticoids, antimetabolites (e.g., hydroxyurea (hydroxycarbamide)), antifolates (e.g., methotrexate), and purine analogs (e.g., azathioprine, mercaptopurine and thioguanine), calcineurin inhibitors (e.g, ciclosporin (cyclosporine A), pimecrolimus and tacrolimus)), inosine-5′-monophosphate dehydrogenase (IMPDH) inhibitors (e.g., mycophenolic acid and derivatives thereof (e.g., mycophenolate sodium and mycophenolate mofetil)), mechanistic/mammalian target of rapamycin (mTOR) inhibitors (e.g., rapamycin (sirolimus), deforolimus (ridaforolimus), everolimus, temsirolimus, umirolimus (biolimus A90, zotarolimus and RTP-801), modulators of sphingosine-1-phosphate receptors (e.g., SIPR1) (e.g., fingolimod), serine C-palmitoyltransferase inhibitors (e.g., myriocin), and analogs, derivatives and salts thereof. The additional therapeutic agents can include corticosteroids/glucocorticoids. The additional therapeutic agents can include inhibitors of pro-inflammatory cytokines or receptors therefor, including but not limited to inhibitors of (e.g., antibodies to) tumor necrosis factor-alpha (TNF-α) (e.g, adalimumab, certolizumab pegol, golimumab, infliximab, etanercept, bupropion and ART-621), inhibitors of (e.g., antibodies to) pro-inflammatory interferons (e.g., interferon-alpha [IFN-α)) or receptors therefor, inhibitors of (e.g., antibodies to) pro-inflammatory interleukins or receptors therefor (e.g., IL-1 (e.g., IL-1α and IL-1β) or IL-1R (e.g., EBI-005 (isunakinra)), IL-2 or IL-2R (e.g., basiliximab and daclizumab), IL-4 or IL-4R (e.g., dupilumab), IL-5 (e.g., mepolizumab) or IL-5R, IL-6 (e.g., clazakizumab, elsilimomab, olokizumab, siltuximab and sirukumab) or IL-6R (e.g., sarilumab and tocilizumab), IL-8 or IL-8R, IL-12 (e.g., briakinumab and ustekinumab) or IL-12R, IL-13 or IL-13R, IL-15 or IL-15R, IL-17 (e.g., ixekizumab and secukinumab) or IL-17R (e.g., brodalumab), IL-18 or IL-18R, IL-20 (e.g., the antibody 7E) or IL-20R, IL-22 (e.g., fezakinumab) or IL-22R, IL-23 (e.g., briakinumab, guselkumab, risankizumab, tildrakizumab SCH-9002221, ustekinumab and BI-655066) or IL-23R, IL-31 or IL-31R (e.g., anti-IL-31 receptor A antibodies such as nemolizumab), IL-33 or IL-33R, and IL-36 or IL-36R), and analogs, derivatives, fragments and salts thereof.

The additional therapeutic agents can include inhibitors of the production of pro-inflammatory cytokines or receptors therefor, including but not limited to inhibitors of the production of TNF-α (e.g., myxoma virus M013 protein, Yersinia YopM, protein, glucocorticoids, immunomodulatory imides, PDE4 inhibitors, p38 MAP kinase inhibitors, inhibitors of TLRs such as TLR7 and TLR9, scrim protease inhibitors (e.g., gabexate and nafamostat), and prostacyclin, carbacyclin and analogs and derivatives thereof (e.g., beraprost, cicaprost, ciprosten, eptaloprost, iloprost and treprostinil)), IFN-α (e.g., alefacept and inhibitors of TLRs such as TLR7 and TLR9), IL-1 (e.g., IL-1α, and IL-1β) (e.g., M013 protein, YopM protein, nafamostat, prostacyclin, glucocorticoids, TNF-α inhibitors, inhibitors of TLRs such as TLR7 and TLR9, and PARI antagonists), IL-2 (e.g., glucocorticoids, calcineurin inhibitors and PDE4 inhibitors), IL-4 (e.g., glucocorticoids and serine protease inhibitors (e.g., gabexate and nafamostat)), IL-5 (e.g., glucocorticoids), IL-6 M013 protein, nafamostat, prostacyclin, tranilast, glucocorticoids, immunomodulatory imides, TNF-α inhibitors, and inhibitors of TLRs such as TLR7 and TLR9), IL-8 alefacept, glucocorticoids and PAR2 antagonists (e.g., tetracyclines)), IL-12 (e.g., apilimod, YopM protein, PDE4 inhibitors, and inhibitors of TLRs such as TLR7 and TLR9), IL-15 (e.g., YopM protein), IL-17 (e.g., protein kinase C (PKC) inhibitors such as sotrastaurin), IL-18 (e.g., MOD protein and YopM protein), and IL-23 (e.g., apilimod, alefacept and PDE4 inhibitors), and analogs, derivatives, fragments and salts thereof.

The additional therapeutic agents can include other kinds of anti-inflammatory agents, including but not limited to inhibitors of pro-inflammatory transcription factors e.g., inhibitors of NE-KB (e.g., nafamostat, M013 protein, penetranin, (−)-DHMEQ, IT-603, IT-901 and PBS-1086) and inhibitors of STAT (signal transducer and activator of transcription) proteins (e.g., JAK1, JAK2 and JAK3 inhibitors)), antagonists of the prostaglandin D2 receptor (DP1) or/and the chemoattractant receptor homologous molecule expressed on TH2 cells (CRTH2) (e.g., TS-022), phosphodiesterase (PDE) inhibitors (e.g., PDE4 inhibitors such as apremilast, cilomilast, ibudilast, piclamilast, roflumilast, crisaborole, diazepam, luteolin, mesembrenone, rolipram, AN2728 and E6005), IgE inhibitors (e.g., anti-IgE antibodies such as omalizumab), myeloperoxidase inhibitors (e.g., dapsone), specialized pro-resolving mediators (SPMs) (e.g., metabolites of polyunsaturated fatty acids such as lipoxins, resolvins (including resolvins derived from 5Z,8Z,11Z,14Z,17Z-eicosapentaenoic acid (EPA), resolvins derived from 4Z,7Z,10Z,13Z,16Z,19Z-docosahexaenoic acid (DHA), and resolvins derived from 7Z, 10Z,13Z,16Z,19Z-docosahexaenoic acid (n-3 DPA), protectins/neuroprotectins (including DHA-derived protectins/neuroprotectins and n-3 DPA-derived protectins/neuroprotectins), maresins (including DHA-derived maresins and n-3 DPA-derived maresins), n-3 DPA metabolites, n-6 DPA (4Z,7Z,10Z,13Z,16Z-docosapentaenoic acid) metabolites, oxo-DHA metabolites, oxo-DPA metabolites, docosahexaenoyl ethanolamide metabolites, cyclopentenone prostaglandins (e.g., Δ12-PGJ2 and 15-deoxy-Δ12,14-PGJ2), and cyclopentenone isoprostanes (e.g., 5,6-epoxyisoprostane A2 and 5,6-epoxyisoprostane E2)), disease-modifying antirheumatic drugs (DMARDs, e.g., sulfasalazine and mesalazine (5-aminosalicylic acid)), anti-allergic agents (e.g., antihistamines, inhibitors of leukotrienes or receptors therefor or the production thereof, mast cell stabilizers, glucocorticoids, epinephrine (adrenaline) and tranilast), ultraviolet radiation (e.g., ultraviolet A and B), and analogs, derivatives, fragments and salts thereof. The additional therapeutic agents can include antagonists of serotonin receptors, antagonists of muscarinic acetylcholine receptors (e.g., MI to M5).

The one or more antiviral agents and/or the one or more additional therapeutic agents can one or more of the following: Gimsilumab, an anti-granulocyte-macrophage colony stimulating factor monoclonal antibody, a non-viral gene therapy producing monoclonal antibodies, EB05, a non-steroidal anti-inflammatory molecule (sPLA2 inhibitor), Opdivo (nivolumab), a PD-1 blocking antibody, IC14, a recombinant chimeric anti-CD14 monoclonal antibody, avastin (bevacizumab), a vascular endothelial growth factor inhibitor, a PD-1 blocking antibody, Thymosin, meplazumab, an anti-CD147 antibody, an antibody combination REGN-COV2 (REGN10933+REGN10987) against the spike protein MEDI3506, a monoclonal antibody targeting interleukin 33, OmniChicken platform antibodies, antibodies from recovered COVID-19 patients, Antibody 47D11, Polyclonal hyperimmune globulin (H-IG), LY-CoV555 antibody, otilimab, an anti-granulocyte macrophase colony-stimulating factor (GM-CSF) antibody, LY3127804, an anti-Angiopoietin 2 (Ang2) antibody, a CXC10 antagonist, polyclonal hyperimmune globulin (H-IG), Octagam, intravenous Immunoglobulin (IVIG), single domain antibodies (sdAbs), an engineered monoclonal antibody derived from camelids, a super-antibody or antibody cocktail to target potential mutations of SARS-COV-2, AiRuiKa (camrelizumab), an anti-programmed cell death protein (PD-1) antibody, Linked nanobody antibody, antibodies from recovered COVID-19 patients, OmniRat platform antibodies, Soliris (eculizumab), a complement inhibitor, CT-P59, Ultomiris (ravulizumab-cwvz), rCIG (recombinant anti-coronavirus 19 hyperimmune gammaglobulin), VIR-7831, VIR-7832, Gamifant (emapalumab), an anti-interferon gamma antibody, leronlimab (PRO 140), an CCR5 antagonist, polyclonal hyperimmune globulin (H-IG), Sylvant (siltuximab), an interleukin-6 targeted monoclonal antibody, Actemra (tocilizumab), an interleukin-6 receptor antagonist, Kevzara (sarilumab), an interleukin-6 receptor antagonist, purified ovine immunoglobulin from immunized sheep, lenzilumab, an anti-granulocyte-macrophage colony stimulating factor antibody, Ilaris (canakinumab), an interleukin-1beta blocker, JS016 antibody, TJM2 (TJ003234), an anti-granulocyte-macrophage colony stimulating factor antibody, COVI-SHIELD antibody cocktail, an antibody targeting the S protein, COVID-EIG plasma, SAB-185, polyclonal hyperimmune globulin (H-IG), IFX-1, an anti-C5a antibody, CERC-002, an anti-LIGHT monoclonal antibody, Remsima (infliximab), an anti-TNF antibody, TY027, a monoclonal antibody targeting SARS-COV-2, IgY-110, an anti-CoV-2 antibody (nasal spray application), mavrilimumab, an anti-granulocyte-macrophase colony-stimulating factor receptor-alpha monoclonal antibody, BDB-100, monocloncal anti-C5a antibody, TZLS-501, an anti-interleukin-6 receptor monoclonal antibody, itolizumab, anti-CD6 IgGI monoclonal antibody, GC5131A, BTL-tml, galidesivir, emetine hydrochloride, DAS181, recombinant sialidase (nebulized), Favilavir/Favipiravir/T-705/Avigan, Vicromax, ISR-50, Levovir (clevudine), AB001, EIDD-2801, an oral ribonucleoside analog, ASC09, an HIV protease inhibitor, Tamiflu (oseltamivir), a neuraminidase inhibitor, Truvada, emtricitabine, tenofovir, a HIV-1 nucleoside analog reverse transcriptase inhibitor, Virazole, ribavirin for inhalation solution, AT-527, an oral purine nucleotide prodrug, Ganovo (danoprevir), a hepatitis C virus NS3 protease inhibitor, ritonavir, remdesivir, a nucleotide analog, Arbidol (umifenovir), Prezcobix (darunavir, HIV-1 protease inhibitor/cobicistat, CYP3A inhibitor), Kaletra/Aluvia (lopinavir/ritonavir), an HIV-1 protease inhibitor, prophylactic antiviral CRISPR in human cells (PAC-MAN), GC376, AmnioBoost, concentrated allogeneic MSCs and cytokines derived from amniotic fluid, Astrostem-V, allogenic adipose-derived mesenchymal stem cells (HB-adMSCs), bone marrow-derived allogenic mesenchymal stem cells (BM-Allo-MSC), mesenchymal stem cells, allogenic adipose-derived mesenchymal stem cells (HB-adMSCs) haNK, natural killer cells, Ryoncil (remestemcel-L), allogenic mesenchymal stem cells, MultiStem, bone marrow stem cells, allogeneic T-cell therapies, Autologous Adipose-Tissue Derived Mesenchymal Stem Cells (ADMSCs) and allogeneic MSCs, CYNK-001, CAP-1002, allogenic cardiosphere-derived cells, PLX cell product, placenta-based cell therapy, Chimeric antigen receptors (CAR)/T cell receptors (TCR)-T cell therapy, natural killer cell-based therapy, small mobile stem (SMS) cells, IMS001, human embryonic stem cell-derived mesenchymal stem cells (hES-MSC), VIR-2703 (ALN-COV) siRNA, OT-101, a TGF-Beta antisense drug, inhaled mRNA, peptide conjugated antisense oligonucleotides, Ampligen, rintatolimod, BXT-25, glycoprotein, EDP1815, Ivermectin, tradipitant, a neurokinin-1 receptor antagonist, piclidenoson, A3 adenosine receptor agonist, Ryanodex (dantrolene sodium), a skeletal muscle relaxant, Jakafi/jakavi (ruxolitinib), nitazoxanide, antiprotozoal, peptides targeting the NP protein, interferon/peginterferon alpha-2b, PegIntron, Sylatron, IntronA, PegiHep, roscovitine seliciclib, cyclin-dependent kinase (CDK) 2/9 inhibitor, ATYR1923, a fusion protein comprising immuno-modulatory domain of histidyl tRNA synthetase fused to the Fc region of a human antibody, a modulator of neuropilin-2, Leukine (sargramostim, rhu-Granulocyte macrophage colony stimulating factor), ADX-1612, HSP 90 inhibitor, DSTAT (dociparstat sodium), glycosaminoglycan derivative of heparin, BIO-11006, Recombinant human interferon alpha-1b, ST-001 nanoFenretinide (fenretinide), Activase (alteplase), tissue plasminogen activator (tPA), camostat mesylate, a transmembrane protease serine 2 (TMPRSS2) inhibitor, nitric oxide, Cozaar (losartan), an angiotensin II receptor blocker (ARB), Otezla (apremilast), an inhibitor of phosphodiesterase 4 (PDE4), IMU-838, a selective oral dihydroorotate dehydrogenase (DHODH) inhibitor, Colchicine, Brilacidin, a defensin mimetic, Metablok (LSALT peptide), a selective dipeptidase-1 antagonist, nafamostat, CD24Fc, an agent comprising nonpolymorphic regions of CD24 attached to the Fc region of human IgGI, Aplidin (plitidepsin), fadraciclib (CYC065), a cyclin-dependent kinase (CDK) 2/9 inhibitor, Aviptadil, a synthetic form of Vasoactive Intestinal Polypeptide (RLF-100), solnatide, a synthetic molecule with a structure based on the lectin-like domain of human Tumour Necrosis Factor alpha, PP-001, MRx-4DP0004, a strain of Bifidobacterium breve isolated from the gut microbiome of a healthy human, ARMS-1, BLD-2660, a small molecule inhibitor of calpain (CAPN) 1, a small molecule inhibitor of CAPN2, a small molecule inhibitor of CAPN9, LAU-7b (fenretinide), N-803, an IL-15 “superagonist” (Nogapendekin alfa inbakicept), Rebif, interferon beta-1a, DIBI, an iron-binding polymer, EPAspire, an oral formulation of highly purified eicosapentaenoic acid free fatty acid (EPA-FFA) in gastro-resistant capsules, MN-166 (ibudilast), a small molecule macrophase migration inhibitory factor (MIF) inhibitor, a phosphodiesterase (PDE) 4 inhibitor, a PDE10 inhibitor, ADX-629, an orally available reactive aldehyde species (RASP) inhibitor, Calquence (acalabrutinib), a Bruton's tyrosine kinase (BTK) inhibitor, Auxora (CM4620-IE), a calcium release-activated calcium (CRAC) channel inhibitor Neumifil, a multivalent carbohydrate binding molecule, Diovan (valsartan), an angiotensin II receptor blocker (ARB), Yeliva (opaganib, ABC294640), an oral sphingosine kinase-2 (SK2) selective inhibitor, WP1122, a glucose decoy prodrug, Kineret (anakinra), an interleukin-1 receptor antagonist, a microbiome therapeutic, Coronzot, bemcentinib, a selective AXL kinase inhibitor, a synthesized nanoviricide drug, Chloroquine/Hydroxychloroquine, an antimalarial drug Senicapoc, vazegepant, a CGRP receptor antagonist, APN01, a recombinant soluble human Angiotensin Converting Enzyme 2, GP1681, a small molecule inhibitor of cytokine release, ST266, a cell-free biologic made from anti-inflammatory proteins secreted by placental cells, recombinant human plasma gelsolin (rhu-pGSN), pacritinib, an oral kinase inhibitor with specificity for JAK2, IRAK1 and CSFIR, Ruconest (recombinant human Cl esterase inhibitor), Cerocal (ifenprodil), NP-120, an NDMA receptor glutamate receptor antagonist targeting Glu2NB, Peginterferon lambda, Pepcid (famotidine), a histamine-2 (H2) receptor antagonist, heparin, a low molecular weight heparin (enoxaparin), an anticoagulant, Xeljanz (tofacitinib), a Janus kinase (JAK) inhibitor, Xpovio (selinexor), a selective inhibitor of nuclear export (SINE) compound, a pH barrier, transepithelial nebulized alkaline treatment, Luvox (fluvoxamine), a selective serotonin reuptake inhibitor, Micardis (telmisartan), brensocatib, a reversible inhibitor of dipeptidyl peptidase 1 (DPP1) Novaferon, RHB-107 (upamostat, WX-671), a serine protease inhibitor, UNI9011, FW-1022, DWRX2003, niclosamide, Lysteda/Cyklokapron/LB1148 (tranexamic acid), an antifibrinolytic PUL-042 inhalation solution, ABX464, Gleevac (imatinib), Traumakine (interferon beta 1-a), Veyonda (idronoxil), Farxiga (dapagliflozin), a sodium-glucose cotransporter 2 (SGLTs) inhibitor, Gilenya (fingolimod), a sphingosine 1-phosphate receptor modulator, sPIF, a synthetic pre implantation factor, SNG001, an inhaled formulation of interferon beta-1α, Methylprednisolone, ciclesonide (Alvesco), hydrocortisone, corticosteroids Olumiant (baricitinib), a Janus kinase (JAK) inhibitor, dipyridamole (Persantine), an anticoagulant, AT-001, an aldose reductase inhibitor, Vascepa (icosapentethyl), a form of eicosapentaenoic acid, OP-101, a dendrimer-based therapy, apabetalone (RVX-208), a selective BET (bromodomain and extra-terminal) inhibitor, Flarin (lipid ibuprofen), Almitrine, VP01, an Angiotensin II Type 2 receptor activator, leflunomide, a pyrimidine synthesis inhibitor, Pulmozyme (nebulised dornase alfa), a recombinant DNase enzyme, AQCH, MSTT1041A (anti-ST2, the receptor for IL-33), UTTR1147A (IL-22-Fc), CIGB-258, FSD-201, ultramicronized palmitoylethanolamide, PB1046, a long-acting sustained release human vasoactive intestinal peptide (VIP) analogue, PTC299, an oral small molecule inhibitor of dihydroorotate dehydrogenase (DHODH), raloxifene (Evista), an estrogen agonist/antagonist, losmapimod, an oral selective p38 mitogen activated protein kinase inhibitor, dutasteride, an anti-androgen, M5049, small molecule capable of blocking the activation of Toll-like receptor (TLR) 7 and TLR8, Eritoran, a TLR-4 antagonist, desidustat, a hypoxia inducible factor prolyl hydroxylase inhibitor, merimepodib, an IMPDH inhibitor, azithromycin, Cenicriviroc, a chemokine receptor 2 and 5 dual antagonist, Firazyr (icatibant), a bradykinin B2 antagonist, Razoprotafib, Tie 2 activating compound (AKB-9778), or any combination thereof.

Antiviral agents provided include, but are not limited to abacavir; acemannan; acyclovir; acyclovir sodium; adefovir; alovudine; alvircept sudotox; amantadine hydrochloride; amprenavir; aranotin; arildone; atevirdine mesylate; avridine; cidofovir; cipamfylline; cytarabine hydrochloride; delavirdine mesylate; desciclovir; didanosine; disoxaril; edoxudine; efavirenz; enviradene; enviroxime; famciclovir; famotine hydrochloride; fiacitabine; fialuridine; fosarilate; trisodium phosphonoformate; fosfonet sodium; ganciclovir; ganciclovir sodium; idoxuridine; indinavir; kethoxal; lamivudine; lobucavir; memotine hydrochloride; methisazone; nelfinavir; nevirapine; palivizumab; penciclovir; pirodavir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin. In certain aspects, other antibodies against viral proteins or cellular factors may be used in combination with a therapeutic composition described herein.

Antibacterial agents provided herein include, but are not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines. Anti-bacterials also include, but are not limited to: Acedapsone, Acetosulfone Sodium, Alamecin, Alexidine, Amdinocillin, Amdinocillin Pivoxil, Amicycline, Amifloxacin, Amifloxacin Mesylate, Amikacin, Amikacin Sulfate, Aminosalicylic acid, Aminosalicylate sodium, Amoxicillin, Amphomycin, Ampicillin, Ampicillin Sodium, Apalcillin Sodium, Apramycin, Aspartocin, Astromicin Sulfate, Avilamycin, Avoparcin, Azithromycin, Azlocillin, Azlocillin Sodium, Bacampicillin Hydrochloride, Bacitracin, Bacitracin Methylene Disalicylate, Bacitracin Zinc, Bambermycins, Benzoylpas Calcium, Berythromycin, Betamicin Sulfate, Biapenem, Biniramycin, Biphenamine Hydrochloride, Bispyrithione Magsulfex, Butikacin, Butirosin Sulfate, Capreomycin Sulfate, Carbadox, Carbenicillin Disodium, Carbenicillin Indanyl Sodium, Carbenicillin Phenyl Sodium, Carbenicillin Potassium, Carumonam Sodium, Cefaclor, Cefadroxil, Cefamandole, Cefamandole Nafate, Cefamandole Sodium, Cefaparole, Cefatrizine, Cefazaflur Sodium, Cefazolin, Cefazolin Sodium, Cefbuperazone, Cefdinir, Cefepime, Cefepime Hydrochloride, Cefetecol, Cefixime, Cefinenoxime Hydrochloride, Cefinetazole, Cefinetazole Sodium, Cefonicid Monosodium, Cefonicid Sodium, Cefoperazone Sodium, Ceforanide, Cefotaxime Sodium, Cefotetan, Cefotetan Disodium, Cefotiam Hydrochloride, Cefoxitin, Cefoxitin Sodium, Cefpimizole, Cefpimizole Sodium, Cefpiramide, Cefpiramide Sodium, Cefpirome Sulfate, Cefpodoxime Proxetil, Cefprozil, Cefroxadine, Cefsulodin Sodium, Ceftazidime, Ceftibuten, Ceftizoxime Sodium, Ceftriaxone Sodium, Cefuroxime, Cefuroxime Axetil, Cefuroxime Pivoxetil, Cefuroxime Sodium, Cephacetrile Sodium, Cephalexin, Cephalexii Hydrochloride, Cephaloglycini, Cephaloridine, Cephalothin Sodium, Cephapirin Sodium, Cephradine, Cetocycline Hydrochloride, Cetophenicol, Chloramphenicol, Cliloramphenicol Palmitate, Chloramphenicol Pantotheniate Complex, Chloramphenicol Sodium Chlorhexidine Succinate, Phosphanilate, Chloroxylenol, Chlortetracycline Bisulfate, Chlortetracycline Hydrochloride, Cinoxacin, Ciprofloxacin, Ciprofloxacin Hydrochloride, Cirolemycin, Clarithromycin, Clinafloxacin Hydrochloride, Clildamycin, Clindamycin Hydrochloride, Clindamycin Palmitate Hydrochloride, Clindamycin Phosphate, Clofazimine, Cloxacillin Benzathine, Cloxacillin Sodium, Cloxyquin, Colistimethate Sodium, Colistin Sulfate, Coumermycin, Coumermycin Sodium, Cyclacillin, Cycloserine, Dalfopristin, Dapsone, Daptomycin, Demeclocycine, Demeclocycine Hydrochloride, Demecycline, Denofungin, Diaveridine, Dicloxacillin, Dicloxacillin Sodium, Dihydrostreptomycin Sulfate, Dipyrithione, Dirithromycin, Doxycycline, Doxycycline Calcium, Doxycycline Fosfatex, Doxycycline Hyclate, Droxacin Sodium, Enoxacin, Epicillin, Epitetracycline Hydrochloride, Erythromycin, Erythromycin Acistrate, Erythromycin Estolate, Erythromycin Ethylsuccinate, Erythromycin Gluceptate, Erythromycin Lactobionate, Erythromycin Propionate, Erythromycin Stearate, Ethambutol Hydrochloride, Ethionamide, Fleroxacin, Floxacillin, Fludalanine, Flumequine, Fosfomycin, Fosfomycin Tromethamine, Fumoxicillin, Furazolium Chloride, Furazolium Tartrate, Fusidate Sodium, Fusidic Acid, Gentamicin Sulfate, Gloximonam, Gramicidin, Haloprogin, Hetacillin, Hetacillin Potassium, Hexedine, Ibafloxacin, Imipenem, Isoconazole, Isepamicin, Isoniazid, Josamycin, Kanamycin Sulfate, Kitasamycin, Levofuraltadone, Levopropylcillin Potassium, Lexithromycin, Lincomycin, Lincomycin Hydrochloride, Lomefloxacin, Lomefloxacin Hydrochloride, Lomefloxacin Mesylate, Loracarbef, Mafenide, Meclocycline, Meclocycline Sulfosalicylate, Megalomicin Potassium Phosphate, Mequidox, Meropenem, Methacycline, Methacycline Hydrochloride, Methenamine, Methenamine Hippurate, Methenamine Mandelate, Methicillin Sodium, Metioprim, Metronidazole Hydrochloride, Metronidazole Phosphate, Mezlocillin, Mezlocillin Sodium, Minocycline, Minocycline Hydrochloride, Mirincamycin Hydrochloride, Monensin, Monensin Sodium, Nafcillin Sodium, Nalidixate Sodium, Nalidixic Acid, Natamycin, Nebramycin, Neomycin Palmitate, Neomycin Sulfate, Neomycin Undecylenate, Netilmicin Sulfate, Neutramycin, Nifuradene, Nifuraldezone, Nifuratel, Nifuratrone, Nifurdazil, Nifurimide, Nifuirpirinol, Nifurquinazol, Nifurthiazole, Nitrocycline, Nitrofurantoin, Nitromide, Norfloxacin, Novobiocin Sodium, Ofloxacin, Ormetoprim, Oxacillin Sodium, Oximonam, Oximonam Sodium, Oxolinic Acid, Oxytetracycline, Oxytetracycline Calcium, Oxytetracycline Hydrochloride, Paldimycin, Parachlorophenol, Paulomycin, Pefloxacin, Pefloxacin Mesylate, Penamecillin, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentizidone Sodium, Phenyl Aminosalicylate, Piperacillin Sodium, Pirbenicillin Sodium, Piridicillin Sodium, Pirlimycin Hydrochloride, Pivampicillin Hydrochloride, Pivampicillin Pamoate, Pivampicillin Probenate, Polymyxin B Sulfate, Porfiromycin, Propikacin, Pyrazinamide, Pyrithione Zinc, Quindecamine Acetate, Quinupristin, Racephenicol, Ramoplanin, Ranimycin, Relomycin, Repromicin, Rifabutin, Rifametane, Rifamexil, Rifamide, Rifampin, Rifapentine, Rifaximin, Rolitetracycline, Rolitetracycline Nitrate, Rosaramicin, Rosaramicin Butyrate, Rosaramicin Propionate, Rosaramicin Sodium Phosphate, Rosaramicin Stearate, Rosoxacin, Roxarsone, Roxithromycin, Sancycline, Sanfetrinem Sodium, Sarmoxicillin, Sarpicillin, Scopafungin, Sisomicin, Sisomicin Sulfate, Sparfloxacin, Spectinomycin Hydrochloride, Spiramycin, Stallimycin Hydrochloride, Steffimycin, Streptomycin Sulfate, Streptonicozid, Sulfabenz, Sulfabenzamide, Sulfacetamide, Sulfacetamide Sodium, Sulfacytine, Sulfadiazine, Sulfadiazine Sodium, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, Sulfas alazine, Sulfasomizole, Sulfathiazole, Sulfazamet, Sulfisoxazole, Sulfisoxazole Acetyl, Sulfisoxazole Diolamine, Sulfomyxin, Sulopenem, Sultamicillin, Suncillin Sodium, Talampicillin Hydrochloride, Teicoplanin, Temafloxacin Hydrochloride, Temocillin, Tetracycline, Tetracycline Hydrochloride, Tetracycline Phosphate Complex, Tetroxoprim, Thiamphenicol, Thiphencillin Potassium, Ticarcillin Cresyl Sodium, Ticarcillin Disodium, Ticarcillin Monosodium, Ticlatone, Tiodonium Chloride, Tobramycin, Tobramycin Sulfate, Tosufloxacin, Trimethoprim, Trimethoprim Sulfate, Trisulfapyrimidines, Troleandomycin, Trospectomycin Sulfate, Tyrothricin, Vancomycin, Vancomycin Hydrochloride, Virginiamycin, and/or Zorbamycin.

Anti-fungal agents provided herein include, but are not limited to, azoles, imidazoles, polyenes, posaconazole, fluconazole, itraconazole, amphotericin B, 5-fluorocytosine, miconazole, ketoconazole, Myambutol (Ethambutol Hydrochloride), Dapsone (4,4′-diaminodiphenylsulfone), Paser Granules ((aminosalicylic acid granules), rifapentine, Pyrazinamide, Isoniazid, Rifadin IV, Rifampin, Pyrazinamide, Streptomycin Sulfate and Trecator-SC (Ethionamide) and/or voriconazole (Vfend™).

### EXAMPLES

Some aspects of the embodiments discussed above are disclosed in further detail in the following examples, which are not in any way intended to limit the scope of the present disclosure.

### Example 1

Antiviral Activity against Epstein Barr Virus

Described in this example are findings that MDL-001 (pipendoxifene), primarily known for its inhibitory actions on RNA-dependent RNA polymerases (RdRp) at Thumb-1 sites, exhibits antiviral activity against Epstein-Barr Virus (EBV).

### Experimental Design and Results

Performed at the University of Alabama-Birmingham, the testing of MDL-001 on EBV utilized the Akata cell line, yielding significant results. MDL-001 demonstrated an EC50 (effective concentration to reduce viral replication by 50%) of 1.80 μM and an EC90 (effective concentration to reduce viral replication by 90%) greater than 2.00 μM. The compound's CC50 (concentration reducing cell viability by 50%) was observed at 5.68 μM, indicating a selectivity index (SI50) of approximately 3 and an SI90 of less than 3. These data points highlight MDL-001's efficacy in inhibiting EBV at relatively low concentrations with minimal cytotoxicity.

### Methods

Reduction of virus-induced cytopathic effect (Primary CPE assay) is described below. Confluent or near-confluent cell culture monolayers of Vero 76 cells (or other appropriate cell line) were prepared in 96-well disposable microplates the day before testing. Cells were maintained in MEM supplemented with 5% FBS. For antiviral assays the same medium was used but with FBS reduced to 2% and supplemented with 50-μg/ml gentamicin. Compounds were dissolved in DMSO, saline or other diluent. Less soluble compounds were vortexed, heated, and/or sonicated, and if they still do not go into solution were tested as colloidal suspensions. The test compound was prepared at four serial log 10 concentrations. Five microwells were used per dilution: three for infected cultures and two for uninfected toxicity cultures. Controls for the experiment consisted of six microwells that were infected and not treated (virus controls) and six that were untreated and uninfected (cell controls) on every plate. A known active drug was tested in parallel as a positive control drug using the same method as is applied for test compounds. The positive control was tested with every test run.

On the testing day, the growth media was removed from the cells and the test compound applied in 0.1 ml volume to wells at 2× concentration. Virus, normally at a titer that will cause >80% CPE, in 0.1 ml volume was added to the wells designated for virus infection. Medium devoid of virus was placed in toxicity control wells and cell control wells. Plates were incubated at 37° C. with 5% CO₂ until marked CPE (>80% CPE for most virus strains) was observed in virus control wells. The plates were then stained with 0.011% neutral red for approximately two hours at 37° C. in a 5% CO₂ incubator. The neutral red medium was removed by complete aspiration, and the cells were rinsed 1× with phosphate buffered solution (PBS) to remove residual dye if needed. The PBS was completely removed, and the incorporated neutral red eluted with 50% Sorensen's citrate buffer/50% ethanol for at least 30 minutes. Neutral red dye penetrates into living cells, thus, the more intense the red color, the larger the number of viable cells present in the wells. The dye content in each well was quantified using a spectrophotometer at 540 nm wavelength. The dye content in each set of wells was converted to a percentage of dye present in untreated control wells using a Microsoft Excel-based spreadsheet and normalized based on the virus control. The 50% effective (EC50, virus-inhibitory) concentrations and 50% cytotoxic (CC50, cell-inhibitory) concentrations were then calculated by regression analysis. The quotient of CC50 divided by EC50 gives the selectivity index (SI) value. Compounds showing SI values>10 were considered active.

### Example 2

### Antiviral Activity Against Hepatitis B Virus

MDL-001 (i.e., pipendoxifene) has been identified as a potential broad-spectrum antiviral agent. A primary hypothesis focuses on its action via the RdRp Thumb-1 site in single-stranded RNA (ssRNA) viruses. However, the antiviral activity observed against the Hepatitis B virus (HBV), a DNA virus, warrants further exploration. This Example details the HBV results, provides insights into the unique structure of the Hepatitis B genome, and discusses the replication mechanisms that can explain the observed antiviral effects.

### HBV Assay Results

The antiviral activity of MDL-001 against HBV (HepAD 38 strain) was evaluated using the AD38 cell line, which supports the replication of HBV. The key findings are summarized as follows: EC50: <0.32 μM, CC50: 20.3 μM, SI50: >63.4 The low EC50 value indicates potent antiviral activity, while the high SI50 suggests that MDL-001 selectively inhibits the virus without causing significant cytotoxicity to the host cells. Also see Table 1 below.

### Hepatitis B Virus Genome and Replication Cycle

HBV is a partially double-stranded DNA virus belonging to the Hepadnaviridae family. Unlike typical DNA viruses, HBV has a unique replication cycle that includes reverse transcription, similar to retroviruses. The HBV genome consists of: relaxed circular DNA (rcDNA) which is partially double-stranded and circular; covalently closed circular DNA (cccDNA), which is formed in the nucleus, acting as a template for transcription; and pre-genomic RNA (pgRNA), which serves as a template for reverse transcription back into DNA.

HBV replication involves the viral polymerase's conversion of pgRNA into rcDNA, which has structural and functional similarities to RNA-dependent RNA polymerases (RdRp) found in ssRNA viruses. This polymerase contains distinct domains, including the fingers, palm, and thumb, which are critical to its function.

### Mechanism of Action Hypothesis

Without being bound by any particular theory, the antiviral activity of MDL-001 against HBV likely stems from its interaction with the viral polymerase. Despite being a DNA virus, HBV's replication process involves a reverse transcriptase activity that shares structural features with the RdRp of ssRNA viruses, particularly the thumb and finger domains. MDL-001, which targets the Thumb-1 allosteric site in RdRp, may similarly inhibit the HBV polymerase, disrupting the reverse transcription process and hindering viral replication.

The observed efficacy of MDL-001 against HBV highlights its potential as a broad-spectrum antiviral agent. HBV's unique replication mechanism, involving a reverse transcriptase with structural similarities to RdRp, provides, without being bound by any particular theory, an explanation for the drug's activity against this DNA virus. These findings not only expand the therapeutic potential of MDL-001 but also underscore the importance of further mechanistic studies to elucidate its antiviral action fully.

TABLE 1
Evaluation of HBV Inhibition in AD38 cells

| | AD38 HBV DNA | | |
|---|---|---|---|
| | EC₅₀ (μM) | TC₅₀ (μM) | TI |
| TDF* | 0.061 | >5.0 | >82.4 |
| MDL-001 | <0.32 | 20.3 | >63.4 |

*Tenofavir disoproxil

### AD38 Assay Methodology

AD38 cells were seeded at a density of 5×10⁴ cells per well in flat-bottomed 96 well plates in the presence of G418 and tetracycline 48 hours prior to the addition of compounds at 37° C./5% CO₂. Plates were washed with DPBS to remove the tetracycline prior to compound addition. Compounds were diluted as indicated in assay media and added to plates in triplicate in a volume of 200 μL. Plates were incubated for an additional 5 days. On day 7 of the assay (post plating of cells), the supernatant was removed and stored at −20° C. until the qPCR endpoint analysis was performed. Assay media was added back to the plate in a volume of 200 μL and the cells were stained with XTT to evaluate cellular toxicity. The optical density of the toxicity cell culture plate was determined spectrophotometrically at 450 and 650 nm using Softmax Pro 5.4.2 software. Total extracellular HBV DNA copy number was evaluated by quantitatively determining extracellular HBV DNA in treated and untreated (virus control) media via qPCR assay, using specifically designed Taqman probe and primer sets.

### Example 3

### Evaluation of HBV Inhibition in AD38 Cells

Provided in this example are further studies of HBV inhibition by pipendoxifene.

### AD38 Assay Methodology

AD38 cells were seeded at a density of 5×10⁴ cells per well in flat-bottomed 96 well plates in the presence of G418 and tetracycline 48 hours prior to the addition of compounds at 37° C./5% CO₂. Plates were washed with DPBS to remove the tetracycline prior to compound addition. Compounds (pipendoxifene) were diluted in assay media and added to plates in triplicate in a volume of 200 μL. Plates were incubated for an additional 5 days. On day 7 of the assay (post plating of cells), the supernatant was removed and stored at −20° C. until the qPCR endpoint analysis was performed. Assay media was added back to the plate in a volume of 200 μL and the cells were stained with XTT to evaluate cellular toxicity. The optical density of the toxicity cell culture plate was determined spectrophotometrically at 450 and 650 nm using Softmax Pro 5.4.2 software. Total extracellular HBV DNA copy number was evaluated by quantitatively determining extracellular HBV DNA in treated and untreated (virus control) media via qPCR assay, using specifically designed Taqman probe and primer sets. Inhibition of HBsAg was evaluated using the methodology described below.

### Anti-HBsAg CLIA Assay Methodology

Culture supernatants were used for analysis of HBs antigen present using Ig Biotechnology HBsAg CLIA kit (Ref CL18002). Fifty microliters (50 μL) of supernatant were incubated with 50 μL enzyme conjugate (HRP labeled anti-HBs MAb in DPBS containing Casein and ProlClin 300) in a 96-well test plate for 60 minutes at 37° C. Following incubation, the contents of the plate were removed, and the plate was washed six times with Wash Solution (DPBS-Tween, 1×). Substrate solution (hydrogen peroxide and luminol in buffer containing enhancers) was prepared and 50 μL was added to each well. The plate was incubated for 10 minutes in the dark, then read using a luminometer (Synergy HT, Bio-Tek) measuring the Relative Light Units (RLU) of each well. Data was analyzed for HBsAg inhibition using MS Excel to calculate IC50 values.

Results are shown in Table 2 below.

TABLE 2
Evaluation of HBV Inhibition in AD38 cells

| | AD38 HBV DNA | | | AD38 HBV HBsAg | | |
|---|---|---|---|---|---|---|
| | EC₅₀ | TC₅₀ | | EC₅₀ | TC₅₀ | |
| | (μM) | (μM) | TI | (μM) | (μM) | TI |
| TDF* | 1.32 | >5.0 | >3.79 | — | — | — |
| MDL-001 | <0.01 | 12 | >1176.47 | 8.35 | 12.0 | 1.44 |
| RG7834 (nM) | — | — | — | 1.15 | >1000 | >869.6 |

*tenofavir disoproxil

### Example 4

### Evaluation of HBV Inhibition by Pipendoxifene

Provided in this Example are further analyses of MDL-001 against HBV and pharmacokinetic studies.

### MDL-001 Against HBV (DNA and HBs Antigen Responses) Under Various Dosing Schedules

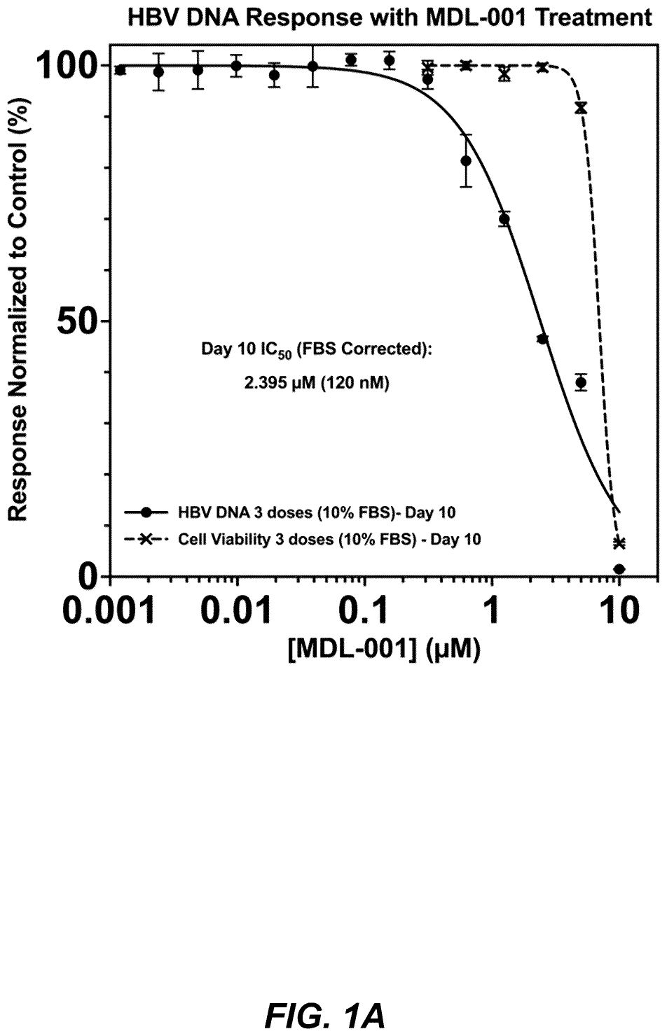

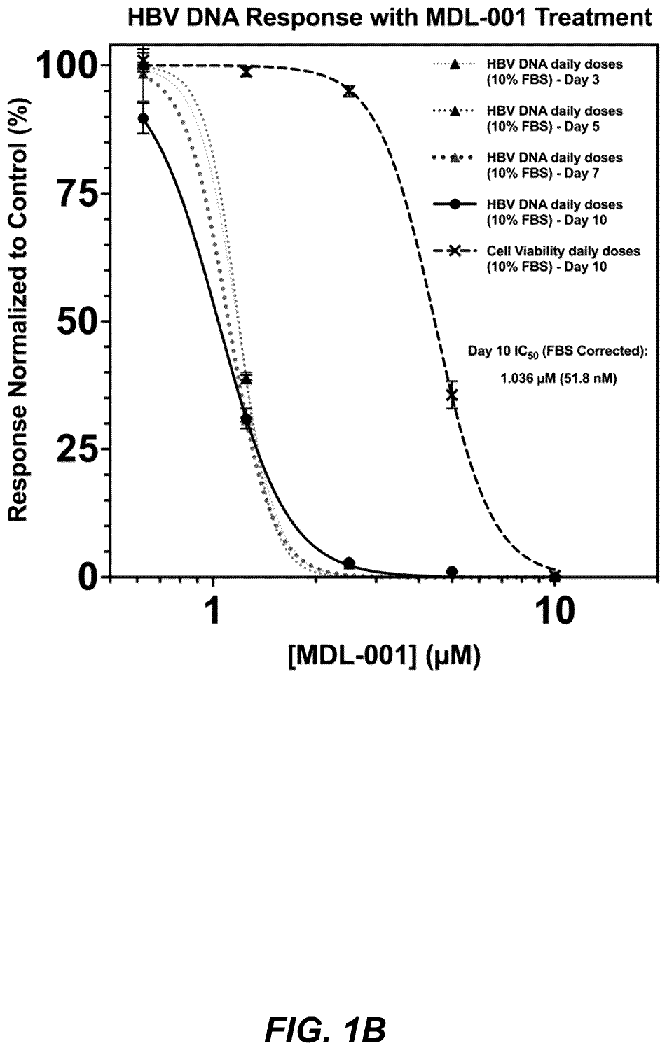

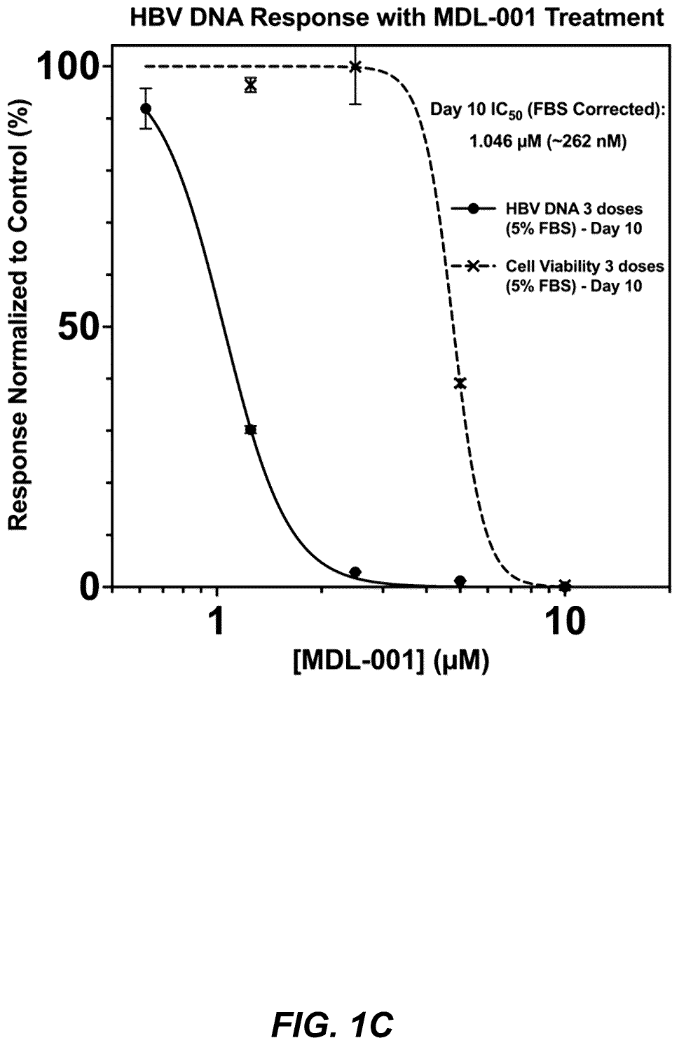

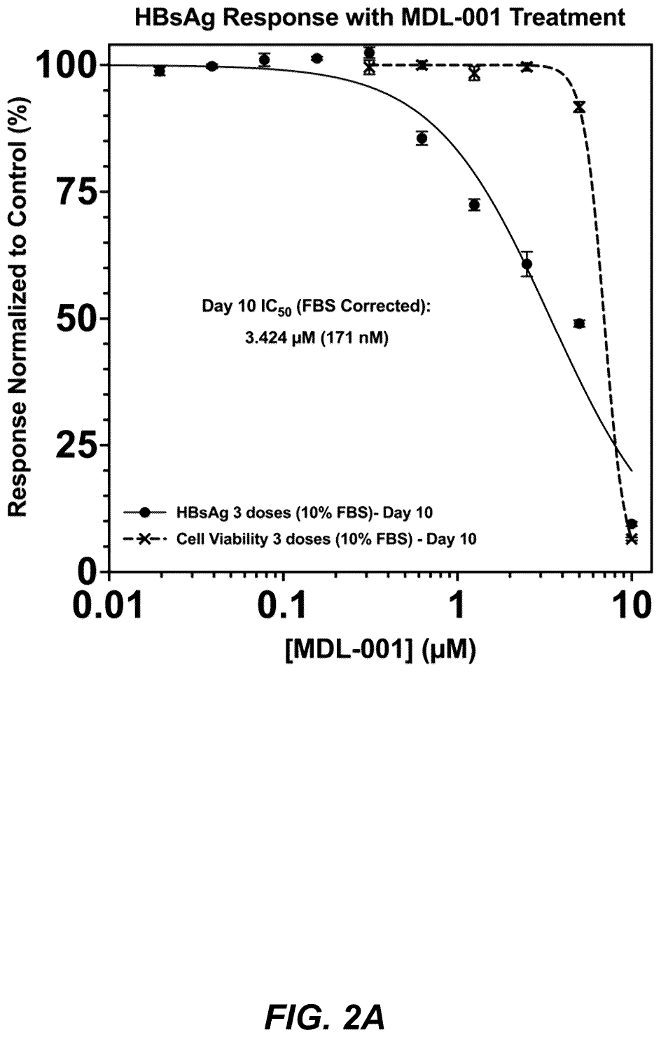

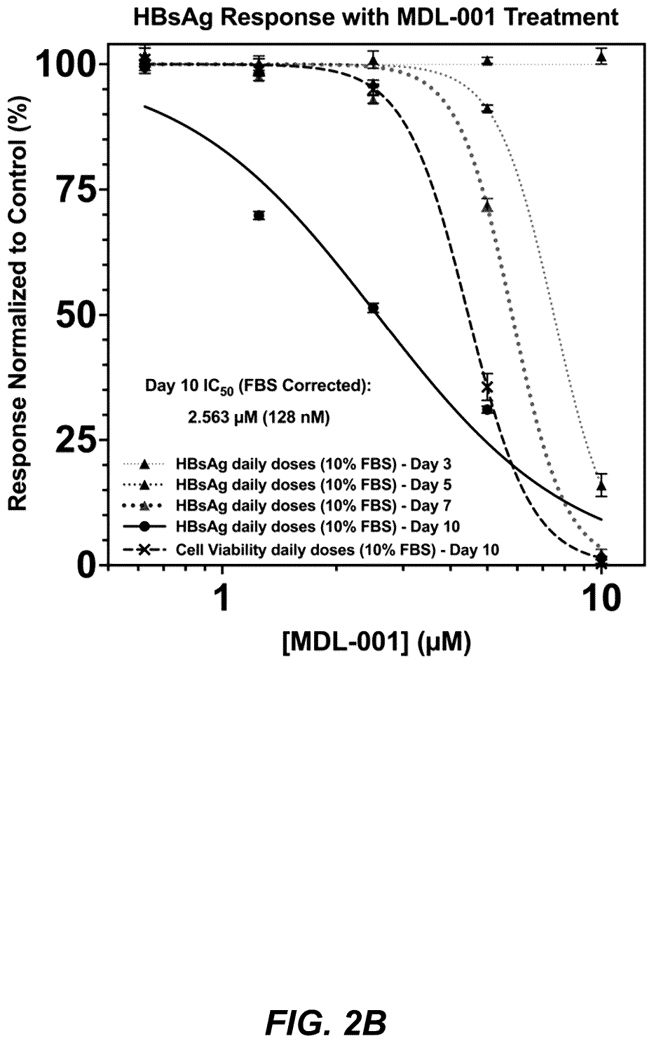

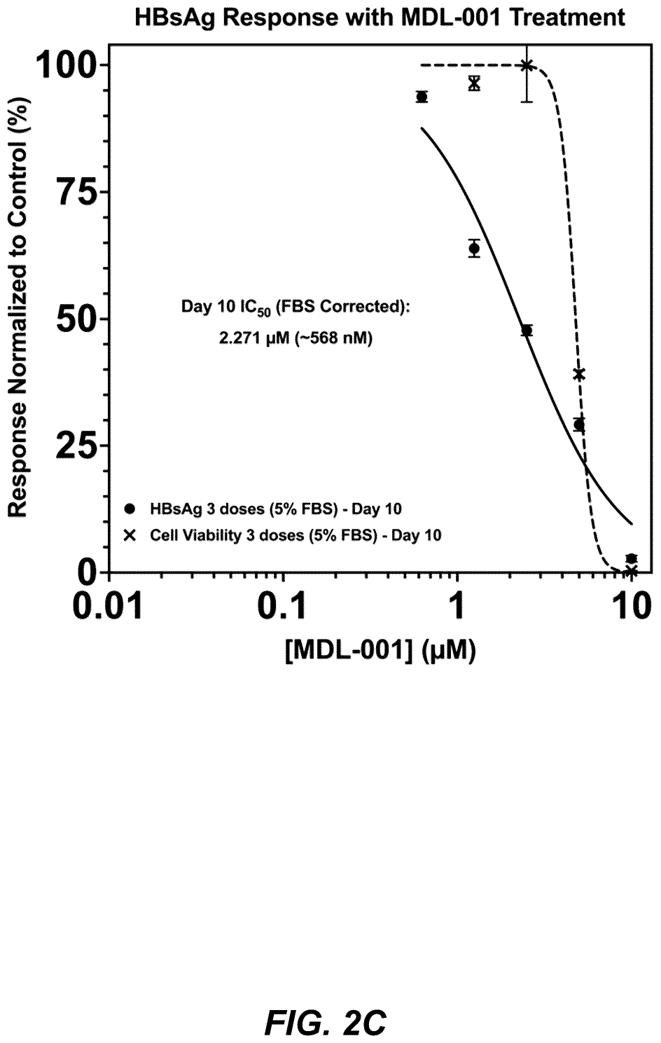

Described below are methods for evaluating the antiviral efficacy of the compound MDL-001 against hepatitis B virus (HBV) in a stably infected cell culture system. Described below are three distinct experimental protocols (FIG. 1A-FIG. 2C).

### Methods

### Experiment 1

A stably HBV-infected HepAD38 cell line was employed to assess MDL-001's inhibitory activity. HepAD38 cells were plated on Day 0 in standard tissue culture-treated vessels. On Day 1, triplicate wells were treated with MDL-001 at concentrations of 0, 1.2, 2.4, 4.9, 9.8, 19.5, 39, 78, 156, 312, 625, 1250, 2500, 5000, and 10000 nM. Drug-containing medium was refreshed on Day 4 and Day 8. On Day 10, supernatants and cell monolayers were harvested for analysis. HBV replication was quantified by HBsAg ELISA and by quantitative PCR of viral DNA in the supernatant. Cell viability was determined using the CellTiter-Glo assay (Promega). Tenofovir alafenamide (TAF) served as a positive control, and DMSO served as a vehicle control.

### Experiment 2

HepAD38 cells were cultured in medium supplemented with 10% fetal bovine serum (FBS). Triplicate wells were treated daily with MDL-001 at concentrations of 0, 0.625, 1.25, and 2.5 μM, beginning on Day 0. Tenofovir alafenamide and DMSO controls were included. Supernatants were collected on Days 3, 5, 7, and 10 for quantification of HBsAg by ELISA and HBV DNA by quantitative PCR. On Day 10, cell viability was assessed using the CellTiter-Glo assay (Promega).

### Experiment 3

HepAD38 cells were maintained in medium containing 5% FBS. Cells were plated on Day 0, and on Day 1 were treated in triplicate with MDL-001 at concentrations of 0, 625, 1250, 2500, 5000, and 10000 nM. Drug treatments were repeated on Day 4 and Day 8. On Day 10, both supernatants and adherent cells were harvested. HBV replication was measured by HBsAg ELISA and quantitative PCR for HBV DNA. Cell cytotoxicity was evaluated by the CellTiter-Glo assay (Promega). Tenofovir alafenamide and DMSO served as positive and vehicle controls, respectively.

### FBS Studies on Pipendoxifene

Protein binding of pipendoxifene was studied using rapid equilibrium dialysis (RED) method as described by van Liempd et al, Development and validation of Higher-Throughput Equilibrium Dialysis Assay for Protein Binding. J. Lab Autom. 16 (1): 56-67 (2011). The protein bound, and unbound fractions observed for pipendoxifene are shown in Table 3.

The protein binding study results showed that pipendoxifene's free concentration in in vitro studies is much lower than its total concentration. Without being bound by any particular theory, it is believed that FBS, which is a significant component of cell assays, lowers the free concentration of pipendoxifene's by 75% at 2% FBS and 95% at 10% FBS. One of the resulting effects is that the observed potency/EC50 numbers can be “normalized” by dividing the EC50 concentration by 20 for the HCV assays (10% FBS) and by 4 for most other assays (2% FBS). Therefore, the results shown below in Table 3 are the “normalized EC50” numbers that are much lower than “observed EC50” numbers. For 2% FBS, an unbound fraction of 25% was observed (and thus a 4× reduction from the apparent EC50), and for 10% FBS, an unbound fraction of 5% was observed (and thus a 20× reduction from apparent EC50).

TABLE 3
Results of protein binding experiments for pipendoxifene, summary of the
mean (n = 3) unbound and bound percentages, and the compound recovery

| | | Conc. | Mean unbound | Mean bound | Std. | Mean |
|---|---|---|---|---|---|---|
| Compound | FBS | (μM) | (F_{ub}, %) | (F_{b}, %) | dev. | recovery (%) |
| Pipendoxifene | 2% heat inactivated | 1 | 25.0 | 75.0 | 3.5 | 106.6 |
| | 10% heat inactivated | 1 | 5.0 | 95.0 | 0.5 | 102.7 |
| | 2% not inactivated | 1 | 24.4 | 75.6 | 4.2 | 91.2 |
| | 10% not inactivated | 1 | 4.7 | 95.3 | 1.1 | 112.4 |

### Analysis of Pipendoxifene (MDL-001) in Mice Plasma, Liver, and Lung Samples

Below in Table 4-Table 6 is a summary of MDL-001 in liver, plasma, and lung of mice following oral administration.

In plasma, near dose-proportional increases in exposure is observed. Significantly lower exposure compared to liver and lung was observed.

TABLE 4
Oral PK of MDL-001 in Mouse Plasma

| | | Dose (mg/kg) | | |
|---|---|---|---|---|
| Parameter | Units | 25 | 50 | 75 |
| Tₘₐₓ | h | 2 | 1 | 1 |
| Cₘₐₓ | ng/mL | 122 | 155 | 253 |
| AUC0-24 h | ng-h/mL | 670 | 932 | 2200 |
| T_{1/2} | h | <8 | NC | 8 |

In liver, near dose-proportional increases in exposure is observed. C_{max, free} at 75 mg/kg is approximately 500 nM.

TABLE 5
Oral PK of MDL-001 in Mouse Liver

| | | Dose (mg/kg) | | |
|---|---|---|---|---|
| Parameter | Units | 25 | 50 | 75 |
| Tₘₐₓ | h | 2 | 1 | 4 |
| Cₘₐₓ | ng/mL | 8636 | 16060 | 19870 |
| AUC0-24 h | ng-h/mL | 43746 | 85461 | 186764 |
| T_{1/2} | h | 8 | 9 | 9 |

19870 ng/m is approximately 500 nM free drug

In lung, dose-proportional increases in Cₘₐₓ; AUC>proportional. C_{max, free} at 75 mg/kg is approximately 336 nM.

TABLE 6
Oral PK of MDL-001 in Mouse Lung

| | | Dose (mg/kg) | | |
|---|---|---|---|---|
| Parameter | Units | 25 | 50 | 75 |
| Tₘₐₓ | h | 2 | 4 | 4 |
| Cₘₐₓ | ng/mL | 4722 | 8136 | 13348 |
| AUC0-24 h | ng-h/mL | 25728 | 67778 | 168258 |
| T_{1/2} | h | 9 | NC | 8 |

13348 ng/m: is approximately 336 nM free drug

### Example 5

### MDL-001 in a Hepatocyte HBV+HCV Co-Infection Model

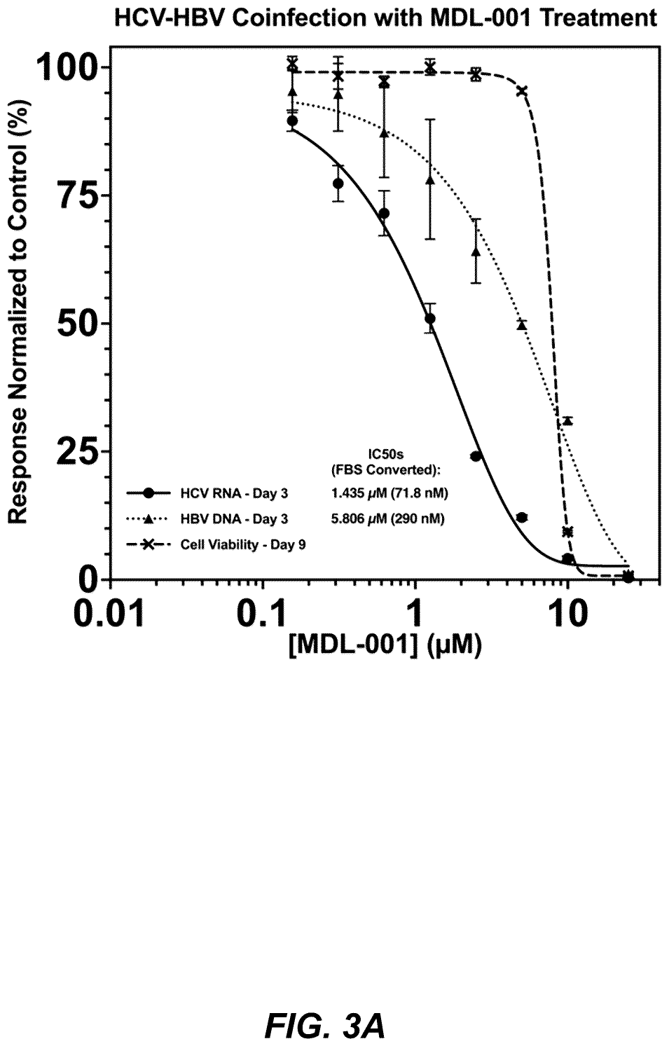

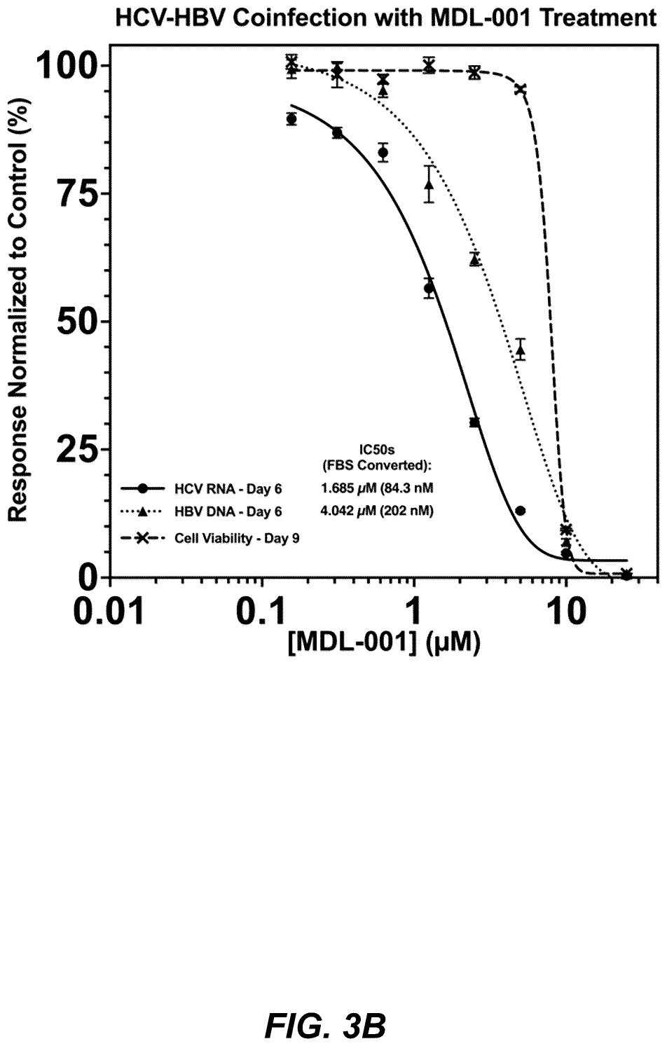

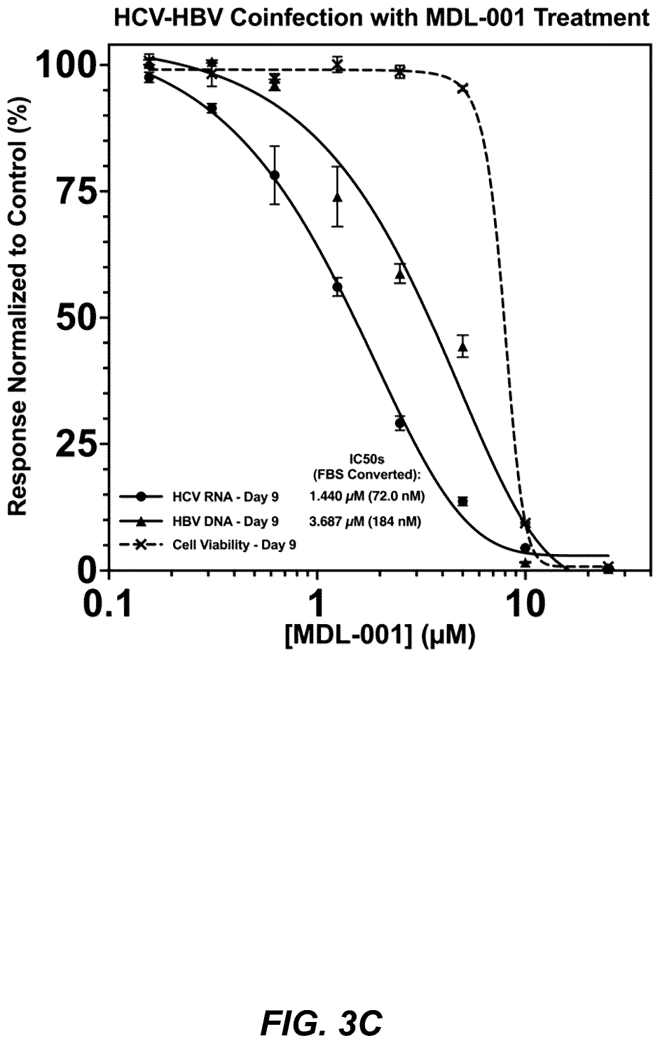

Provided in this Example is evaluation of the antiviral activity of MDL-001 in a hepatocyte co-infection model using Huh7.5.1 cells simultaneously infected with hepatitis C virus (JFH-1 strain) and HBV. Results are shown in FIG. 3A-FIG. 3C.

### Methods

Huh7.5.1 human hepatoma cells were plated in 96-well tissue-culture-treated plates in Dulbecco's Modified Eagle Medium (DMEM) supplemented with 10% fetal bovine serum (FBS) and 1% penicillin/streptomycin. Cells were allowed to attach overnight at 37° C. in a 5% CO₂ atmosphere.

On Day 0, cells were co-infected with hepatitis C virus (HCV JFH-1) and HBV by adding virus stocks directly to each well. After a 2-hour adsorption period at 37° C., inocula were removed, cells were washed twice with phosphate-buffered saline (PBS), and fresh DMEM+10% FBS was added.

Triplicate wells were treated with MDL-001 at final concentrations of 0, 156, 312, 625, 1250, 2500, 5000, 10000, and 25000 nM. Dosing occurred on Day 0 immediately post-infection, and was repeated on Days 3 and 6. Vehicle (DMSO) and antiviral controls remdesivir (for HBV) and sofosbuvir (for HCV) were included at their respective EC50 concentrations.

Supernatants were harvested on Days 3, 6, and 9, clarified by centrifugation (800×g, 5 min), and stored at −80° C. Viral nucleic acids were quantified by one-step RT-qPCR for HCV RNA and qPCR for HBV DNA, using virus-specific primer/probe sets. All values were normalized to the DMSO control.

Cell viability and cytotoxicity were assessed on Day 9 using the CellTiter-Glo luminescent assay (Promega) according to the manufacturer's instructions. Viability is reported as a percentage of DMSO-treated controls; all treatments were performed in triplicate and data are presented as mean±SD.

### Example 6

### MDL-001 In Vivo Study

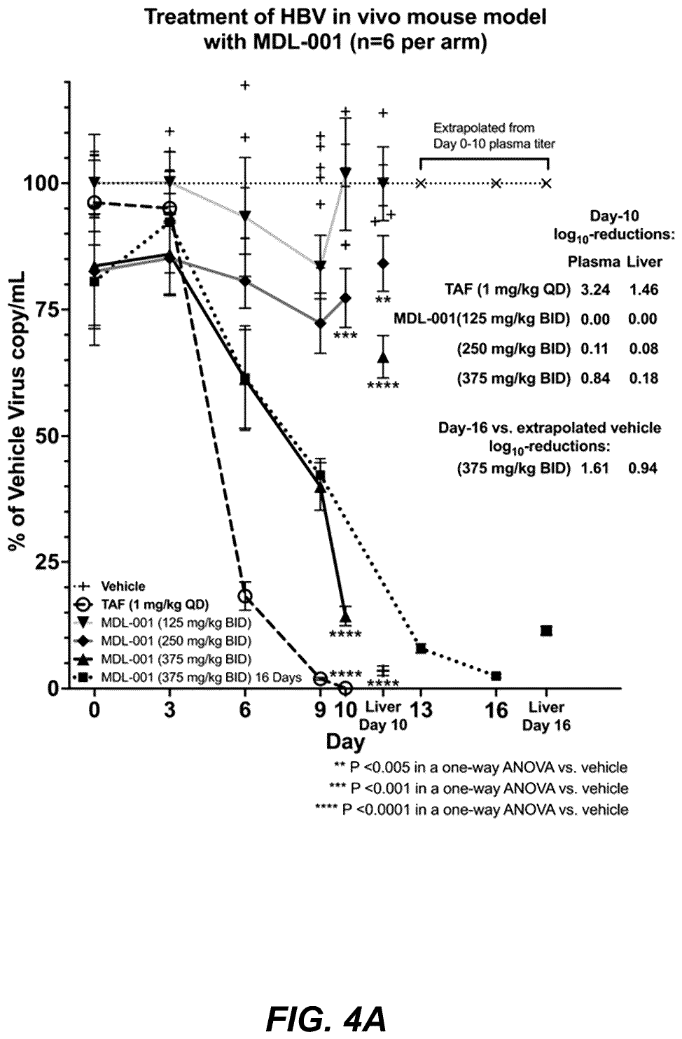

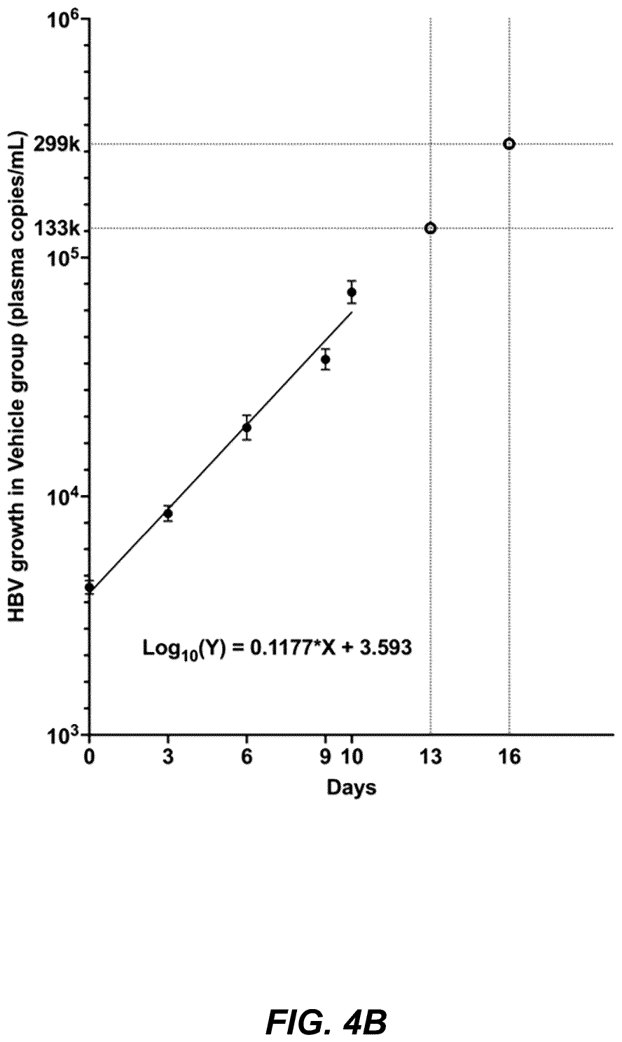

MDL-001's efficacy against HBV was assessed in the humanized MUP-uPA-SCID/Beige mouse model using HBV AD38. Six treatment arms were evaluated: vehicle, positive control, MDL-001 at 125, 250 or 375 mg/kg BID over ten days, plus an extended 375 mg/kg BID cohort through sixteen days. Antiviral effects were measured via serial serum HBV DNA levels and terminal intrahepatic viral burden. Results are shown in FIG. 4A-FIG. 4B.

### Methods

### Animal Care

Animal housing: individually ventilated cage (IVC) racks were used to house the majority of mice. HEPA-filtered air was supplied into each cage at a rate of 60 air changes per hour. Mice were housed in solid bottom cages. Static mouse cages were changed at least once a week. IVCs were changed at least once every 14 days. Certain strains of rodents were changed into clean cages more frequently as needed.

The room environment is described below. Each animal room is equipped with a high/low thermo-hygrometer and its own computerized controlled thermostat. Animal care staff monitored and recorded animal room high/low temperatures and humidity daily on the room activity log. For temperature, alarm points were set at ±4° F. from guidelines. High or low temperature alarms were annunciated to the engineer on duty 24 hours a day. The Department of Animal Resources (DAR) management was notified of excursions. Most of the animal facilities are also equipped with an Edstrom Industries Watchdog environmental monitoring system in addition to the automated building management system (BMS). The Watchdog system registers temperature and humidity, and also sends alarms to Animal Resources management personnel. Humidity levels were not controlled in any of the facilities but are reliably maintained between 30-70% most of the year.

Details of diet are described below. Food (Teklad LM-485 autoclavable diet) was provided ad libitum to mice in wirebar lids. The vivarium is equipped with a reverse osmosis (R/O) water purification system and automatic watering distribution system from Edstrom Industries. DAR receives monthly water quality reports from the City of San Diego. R/O purified water was monitored daily during the workweek. A number of parameters were monitored including conductivity, temperature, pH level and chlorine concentration. Automatic water delivery systems (room and rack distribution lines) were timed for daily in-line flushing. Quick disconnect drinking valves were sanitized with each cage change or more often if needed. System sanitation and preventive maintenance was performed by the DAR equipment technicians.

Details of acclimation period are described below. Mice were allowed up to 72 hours to stabilize into their new housing environment. Some experimental paradigms involve examining the behavioral response to novelty and therefore the animal cannot be habituated to the procedure.

In order to minimize suffering, all surgical procedures were carried out under anesthesia using isoflurane (1-4%) in conjunction with ketamine/xylazine ip (90 120 mg/Kg and 10 mg/Kg). Mice were monitored every 15 minutes after induction for respiratory and heart rates if the surgical procedure required more time. Animals were provided buprenorphine (0.05-2.5 mg/Kg s.c.) for 6-12 h followed by flunixine meglumine (2.5 mg/Kg s.c.) as a postoperative analgesic for 2 days post-implantation. Mice were observed 2 h, 6 h and 24 h post-surgery with daily monitoring during the course of the study. Mice were supplied with acidified water supplemented with sulfamethoxazole (or, sulfadiazine) with trimethoprim at a final concentration of 0.65-1.6 mg/mL to reduce chances of opportunistic bacterial colonization. MUP-uPA-SCID/Beige mice were maintained at DAR at TSRI in accordance with protocols approved by the TSRI Ethics Committee, the Institutional Animal Care and Use Committee (Protocol Number: 11-0015). This study was carried out in strict accordance with the recommendations in the Guide for the Care and Use of Laboratory Animals of the National Institutes of Health. All efforts were made to minimize suffering. The method of sacrifice used for the experimental mice will be cervical dislocation. A power calculation will be used to determine the sample size (number of mice/group). 10 mice per group were used for each treatment in all experiments.

### HBV Chimeric Mouse Study

Transgenic mice carrying the uPA gene driven by the major urinary protein promoter were crossed onto a SCID/Beige background (MUP-uPA-SCID/Beige). These transgenic mice are healthier than former Alb-uPA mice and provide an extended window from age 4 to 1 months for engraftment with human hepatocytes and infection with viral hepatitis viruses derived from serum of infected chimpanzees or from concentrated supernatant of HBV-replicating cell culture. MUP-uPA-SCID/Beige mice (gift from A. Kumar) (4 months old) were transplanted with human hepatocytes (10e7 cells/mouse) (˜300-500×10e6 hepatocytes per donor are usually obtained, gift from D. Geller). In brief, fresh hepatocytes were transplanted immediately upon arrival within 12-16 hour after isolation. Viable cell counts were determined. 1 cm skin incision were made in the upper left quadrant of the mouse abdomen to visualize the spleen. Human hepatocytes were injected intra-splenically. Incision was closed with Vetbond tissue adhesive (3M Animal Care Products, St. Paul, MN). To verify the degree of “humanization”, blood was weekly collected for human albumin quantification by ELISA (Bethyl Laboratories) according to the manufacturer's protocol. Mice expressing >300 μg/mL of human albumin were randomized into groups of 10 mice. Note that the expression of the uPA transgene causes damage to the murine liver that is rapidly replaced by clusters of implanted human hepatocytes that can be visualized by human albumin immunostaining. MUP-uPA-SCID/Beige mice that had been engrafted with human hepatocytes were infected intravenously (i.v.) with concentrated HBV AD38 virus derived from cell culture. On Day 0, humanized mice (n=6 per arm; 36 total) were infected via tail-vein injection with 1×10\hat{ }7 genome equivalents of HBV AD38. Groups received oral gavage BID (08:00 and 20:00 h) as follows: vehicle formulation buffer; positive control (entecavir 1 mg/kg once daily); MDL-001 at 125, 250 or 375 mg/kg BID through Day 9; and the extended cohort receiving MDL-001 at 375 mg/kg BID through Day 15. Blood was sampled on Days 3, 6 and 9 for all groups, and on Days 13 and 16 for the extended cohort. Serum was stored at −80° C. and HBV DNA quantified by acid guanidinium-phenol-chloroform extraction followed by TaqMan RT-qPCR. Mice in the five ten-day arms were euthanized on Day 10; the extended cohort was euthanized on Day 16. Livers were collected for HBV DNA analysis, and lungs were snap-frozen for downstream studies.

### Example 7

### MDL-001 Treatment of HBV and HDV Co-Infection

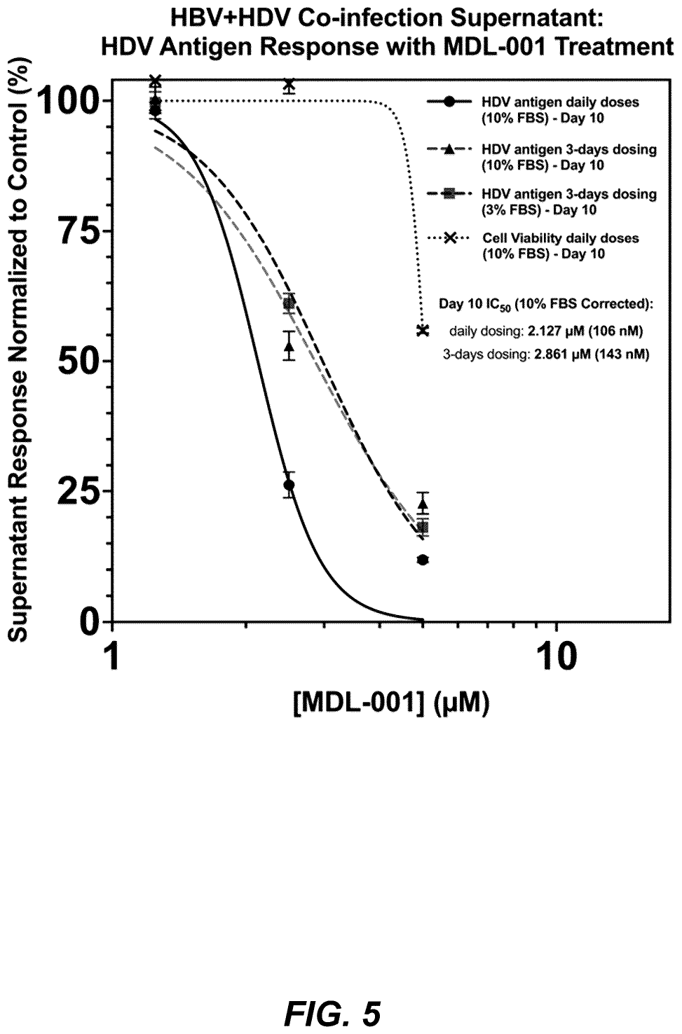

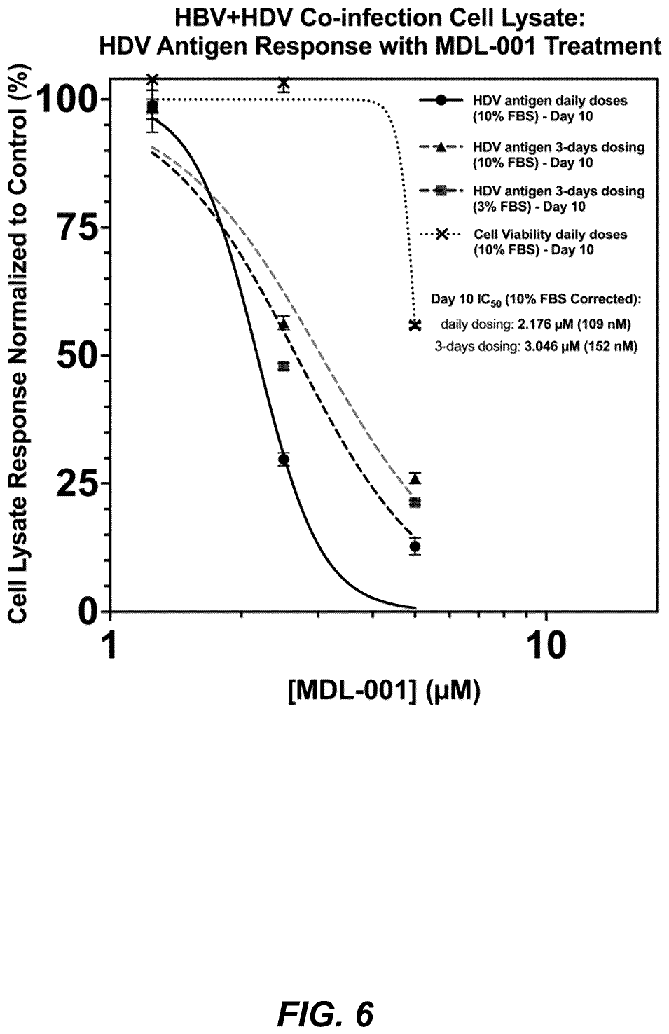

Hepatitis D virus (HDV) is a defective RNA virus requiring hepatitis B virus envelope proteins for assembly and entry. HDV infection accelerates liver disease progression in chronically HBV-infected patients and few targeted therapies exist. Described in this Example is the antiviral activity of MDL-001 in an in vitro HDV infection model using Huh7 cells expressing the sodium taurocholate cotransporting polypeptide (hNTCP-Huh7). By quantifying intracellular and extracellular HDV antigen levels following single and intermittent dose regimens under varying serum conditions we sought to characterize the potency and dosing kinetics of MDL-001 relative to vehicle control. Results are shown in FIG. 5-FIG. 6

### Methods

HDV NTCP+ Huh7 Cells MDL-001

### HDV Production and Infection

HDV ribonucleoproteins were generated by co-transfection of Huh7 cells with a cytomegalovirus-driven head-to-tail dimer of genotype I HDV cDNA and an HBV envelope-encoding pUC18 plasmid (nucleotides 2431-1990; GenBank U95551.1) as described previously. Supernatants were harvested on day 7 post-transfection to avoid mechanical disruption of virions, clarified by low-speed centrifugation, aliquoted, and stored at −80° C. For infection, hNTCP-Huh7 cells were inoculated at 100 multiplicities of genome equivalents per well in the presence of 4% polyethylene glycol 8000 to enhance uptake.

### Antiviral Assays

hNTCP-Huh7 cells were seeded in triplicate in 96-well plates at 1×10\hat{ }5 cells per well in Williams E medium supplemented with transferrin (5 μg/mL), EGF (10 ng/mL), insulin (3 μg/mL), L-glutamine (2 mM), hydrocortisone (18 μg/mL), dexamethasone (40 ng/mL), sodium selenite (5 ng/ml), 2% dimethyl sulfoxide, penicillin (100 U/mL) and streptomycin (100 μg/mL). On day 1, cells were inoculated with HDV and immediately treated with MDL-001 (2.5 μM), bulevirtide control (2.5 μM), or DMSO vehicle (≤0.1% v/v).

Three dosing regimens were evaluated under 10% or 3% fetal bovine serum conditions. Condition 1 included daily dosing (10% FBS) Drugs were added once per day on days 1 through 9. Cells were lysed on days 3, 5, 7 and 10 and HDV antigen measurement in cell lysates by ELISA (mybiosource.com/hdv-ag-human-elisa-kits/hepatitis-d-virus-hdv-antigen/931694). Cell viability was measured on day 10 using the CellTiter-Glo assay (Promega).

Condition 2 included three-day dosing (10% FBS). Drugs were added on days 1, 4 and 8. Cells were lysed on day 10 for HDV antigen quantification and viability assessment via CellTiter-Glo.

Condition 3 included three-day dosing (3% FBS). Same as regimen 2 but in medium containing 3% FBS. HDV antigen quantification and cytotoxicity assays were performed on day 10.

All data represent the mean of triplicate wells and were normalized to vehicle controls to determine percent inhibition.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for preventing, delaying the onset of, or treating an infection or a disease caused by a deoxyribonucleic acid (DNA) virus, the method comprising administering to a subject in need thereof a composition comprising pipendoxifene or a pharmaceutically acceptable salt, ester, solvate, stereoisomer, tautomer, or prodrug thereof, thereby preventing, delaying the onset of, or treating the infection or the disease, wherein pipendoxifene has a structure of:

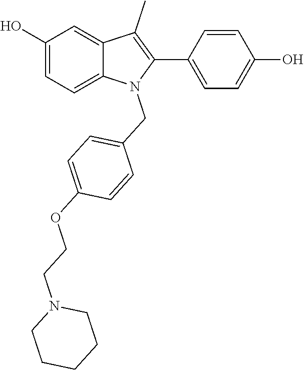 [SMILES: Cc1c(-c2ccc(O)cc2)n(Cc2ccc(OCCN3CCCCC3)cc2)c2ccc(O)cc12]

2. The method of claim 1, wherein the subject has been or is suspected to have been exposed to, or is at risk of exposure to, the DNA virus.

3. The method of claim 1, wherein the subject is suffering from or at risk of the infection or the disease caused by the DNA virus.

4. The method of claim 1, wherein the infection is in the liver or the B cells of the subject.

5. The method of claim 1, wherein the infection or the disease comprises a liver disease, pneumonia, interstitial lung disease, pancreatitis, myocarditis, mononucleosis, cancer, systemic lupus erythematosus (SLE), Sjögren's syndrome, rheumatoid arthritis, or multiple sclerosis (MS).

6. The method of claim 1, wherein the subject is a mammal.

7. The method of claim 1, wherein the DNA virus is a double-stranded DNA virus.

8. The method of claim 1, wherein the composition is in the form of powder, pill, tablet, microtablet, pellet, micropellet, capsule, capsule containing microtablets, liquid, aerosols, or nanoparticles.

9. The method of claim 1, wherein the administering comprises intravenous administration, nasal administration, pulmonary administration, oral administration, parenteral administration, or nebulization.

10. The method of claim 1, wherein administrating the composition reduces or inhibits viral replication of the DNA virus in the subject.

11. The method of claim 1, wherein administering the composition reduces the amount of a viral DNA encoded by the DNA virus in the subject or the amount of an antiviral antibody against the DNA virus in the subject by at least 10%, compared to absence of the administration.

12. The method of claim 1, wherein the subject has been or is suspected to have been exposed to, or is at risk of exposure to, a co-infecting virus, wherein the co-infecting virus comprises a hepatitis C virus (HCV) or a hepatitis D virus (HDV).

13. The method of claim 1, wherein the DNA virus is hepatitis B virus (HBV) or Epstein-Barr virus (EBV).

14. The method of claim 13, wherein administrating the composition reduces or inhibits a viral activity of the co-infecting virus in the subject.

15. A method of inhibiting viral activity, comprising contacting a cell or tissue with a composition that comprises pipendoxifene having a structure of: 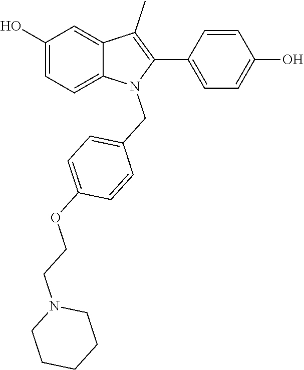 [SMILES: Cc1c(-c2ccc(O)cc2)n(Cc2ccc(OCCN3CCCCC3)cc2)c2ccc(O)cc12]
or a pharmaceutically acceptable salt, ester, solvate, stereoisomer, tautomer, or prodrug thereof, thereby reducing or inhibiting a viral activity of a deoxyribonucleic acid (DNA) virus in the cell or tissue.

16. The method of claim 15, wherein the viral activity is viral replication.

17. The method of claim 15, wherein the contacting occurs in vitro

18. The method of claim 15, wherein the contacting occurs in vivo.

19. The method of claim 15, wherein the DNA virus is a hepatitis B virus (HBV) or an Epstein-Barr virus (EBV).

20. The method of claim 15, wherein the cell or tissue is exposed to a co-infecting virus, wherein the co-infecting virus is a hepatitis C virus (HCV) or a hepatitis D virus (HDV).
